(19) United States
(12) Patent Application Publication
(10) Pub. No.: US 2026/0199259 A1
(43) Pub. Date: Jul. 16, 2026

(54) CONTROLLED LIPID SELF-ASSEMBLY FOR SCALABLE MANUFACTURING OF NEXTGENERATION IMMUNE STIMULATING COMPLEXES

(71) Applicant: MASSACHUSETTS INSTITUTE OF TECHNOLOGY, Cambridge, MA (US)

(72) Inventors: Darrell IRVINE, Arlington, MA (US); Paula HAMMOND, Newton, MA (US); Ivan Susin PIRES, Cambridge, MA (US)

(21) Appl. No.: 19/129,308

(22) PCT Filed: Sep. 14, 2023
(86) PCT No.: PCT/US2023/074206

§ 371 Date: May 13, 2025

Related U.S. Application Data
(60) Provisional application No. 63/383,854, filed on Nov. 15, 2022.

(51) Int. Cl. A61K 9/51

(52) U.S. Cl. CPC A61K 9/5192; A61K 9/5123; A61K 9/5153

(57) ABSTRACT

Methods of controlled lipid self-assembly for scalable manufacturing of next-generation immune stimulating complexes (ISCOMs) are described. The methods involve mixing one or more saponins, one or more lipids, one or more sterols, one or more additional adjuvants (e.g., TLR4 agonist), and optionally one or more antigens in the presence of a detergent. Typically, the mixing step is followed by an incubation step, a dilution and/or concentration step, and/or a filtration or dialysis step for removing detergent. Preferably, methods involve a tangential flow filtration (TFF) process suitable for scalable synthesis and Good Manufacturing Practice (GMP) production of highly homogeneous clinical-grade ISCOM-based adjuvants.

### CROSS-REFERENCE TO RELATED APPLICATIONS

This application claims the benefit of and priority to U.S. Provisional Application No. 63/383,854 filed Nov. 15, 2022, which is hereby incorporated by reference in its entirety.

### STATEMENT REGARDING FEDERALLY SPONSORED RESEARCH

This invention was made with government support under AI161818, AI048240, AI14446, and CA235375 awarded by the National Institutes of Health, W81XWH-13-1-0151 awarded by the Army Medical Research and Development Command, and W911NF-18-2-0048 awarded by the U.S. Army Research Office. The government has certain rights in the invention.

### FIELD OF THE INVENTION

The invention is generally in the field of making nanocage adjuvant particles, methods of use, and the products thereof.

### BACKGROUND OF THE INVENTION

Immune stimulating complexes (ISCOMs) are nanoparticles generated by the self-assembly of cholesterol and phospholipids with saponins. Saponins are triterpene glycosides typically isolated from the soapbark tree Quillaja saponaria Molina that have strong immunostimulatory properties (B. Morein, K. L. Bengtsson, Immunol. Cell Biol. 1998, 76, 295). The resulting 30-40 nm diameter particles have a cage-like structure that have potent activity as vaccine adjuvants in preclinical animal models and humans. ISCOMs were initially studied by assembling saponin/lipids in the presence of antigens in order to embed antigen within the particles, but it was later found that such physical linkage with antigens was not necessary for adjuvant activity. This finding led to use of ISCOM matrices (hereafter, ISCOMs for simplicity) as adjuvants mixed with antigens that are not physically associated to the particles, which have undergone extensive preclinical and clinical development as vaccine adjuvants over the past 30 years (K. Lövgren Bengtsson, B. Morein, A. D. Osterhaus, Expert Rev. Vaccines 2011, 10, 401). In 2021, the European Medicines Agency (EMA) granted a conditional marketing authorization to Nuvaxovid (NVX-CoV2373), a COVID-19 vaccine comprised of SARS-CoV-2 spike glycoprotein nanoparticle antigen mixed with an ISCOM adjuvant termed Matrix-M (L. M. Dunkle, et al., N. Engl. J. Med. 2022, 386, 531). Currently, Nuvaxovid is used by more than 35 countries. ISCOM-adjuvanted vaccine formulations are also in current clinical development for various other viral infections such as seasonal influenza and respiratory syncytial virus (RSV) (C. Keech, et al., N. Engl. J. Med. 2020, 383, 2320). Aiming to improve adjuvant activity with ISCOMs, generation of ISCOM particles incorporating an additional innate immune activator, monophosphoryl lipid A (MPLA), a Toll-like receptor 4 agonist was recently reported. These ISCOM particles, which were termed Saponin-MPLA NanoParticles (SMNP), have enhanced adjuvant activity, making them promising next-generation vaccine adjuvants (M. Silva, et al., Sci. Immunol. 2021, 6).

Several methods have been described in the literature for the manufacture of ISCOMs. The commonly employed approach is to solubilize lipids, cholesterol, and saponin in a high concentration of a non-ionic detergent (typically MEGA-10), followed by detergent removal via ultracentrifugation or dialysis. While dialysis is a simple method leading to uniform ISCOM particle formation, the procedure generally requires more than three days (H. X. Sun, Y. Xie, Y. P. Ye, Vaccine 2009, 27, 4388; International Publication No. WO 1996/011711). Further, neither dialysis nor ultracentrifugation are readily amenable to large-scale manufacturing. More recently, lipid film hydration, ethanol injection and ether injection methods have been developed aimed at improving the scalability of ISCOM processing (H. L. Pham, P. N. Shaw, N. M. Davies, Int. J. Pharm. 2006, 310, 196; H. L. Pham, et al., Current Drug Delivery 2006, 3, 389-397). However, these methods have either led to more heterogeneous formulations, require prolonged exposure of saponin to high temperatures, or have only been employed in hydrophilic saponin fractions.

It is an object of the invention to provide improved methods for making nanocage particles with increased yield compared to existing methods.

It is another object of the invention to provide improved methods for making nanocage particles that are readily amenable to large-scale manufacturing.

It is yet another object of the invention to provide methods for preparing nanocage particles for safe and effective long-term storage with minimal loss in adjuvant activities.

### SUMMARY OF THE INVENTION

Methods of making non-liposome, non-micelle nanocage particles are provided. The methods typically include controlled disassembly of detergent micelles into monomers such that nanocages can self-assembly. The methods can include (a) solubilizing and/or mixing one or more components of lipids, cholesterol, saponin and optionally an additional adjuvant, in the presence of a detergent. Disassembly of detergent micelles can then be carried out. In some embodiments, the methods include (b) reducing the concentration of the detergent to achieve a desired concentration of the detergent. Next, the methods can include (c) removing the detergent from the mixture to promote self-assembly of the one or more components of lipids, cholesterol, saponin and optionally an additional adjuvant into a dispersion of nanocage particles.

Disassembly of detergent micelles such as by reducing the concentration of detergent can be accomplished through a variety means including, but not limited to, dilution of the mixture, increasing ionic strength of the mixture, changing the temperature of the solution, adding one or more detergent-depleting agents, and combinations thereof to achieve a desired concentration of the detergent before the step (c).

For example, in some embodiments, the mixing step is followed by a dilution step, an incubation step, and/or concentration step, prior to the step of removing detergent. In some embodiments, the methods involve a step of dilution of the mixture of (a) to achieve a desired concentration of the detergent before the step (c). In some embodiments, the dilution step is a rapid dilution step achieving a desired concentration of the detergent in less than 30 minutes, less than 20 minutes, less than 10 minutes, or less than 5 minutes, or less than one minute. In other embodiments, the dilution step is a continuous dilution step achieving a desired concentration of the detergent adding buffer dropwise to the solution over a period of between about 1 hour and about 24 hours, for example, at a rate to achieve 10-fold dilution per hour for 10 hours. In preferred embodiments, the dilution step is a staggered discontinuous dilution step achieving a desired concentration of the detergent via stepwise addition of buffer, thus, after each step of adding buffer, the mixture is allowed to equilibrate prior to the next step of adding buffer. In one embodiment, the staggered discontinuous dilution step is performed diluting in 10-fold steps relative to the initial volume and allowing samples sufficient time to equilibrate mixture, before performing the next dilution step. In some embodiments, the methods dilute the mixture to about or below the critical micelle concentration (CMC) of the detergent. In some embodiments, the methods involve an incubation step after step (b) and/or after the dilution step, prior to before step (c), preferably, for an effective amount of time for the formation of nanocages, for example, between about 10 minutes and about 24 hours.

The step of removing the detergent from the mixture can be carried out via dialysis, centrifugation, filtration, or combinations thereof, optionally using a filtration membrane with a molecular weight cutoff (MWCO) between about 10 kDa and about 100 kDa, inclusive. In preferred embodiments, the detergent is removed from the mixture via filtration such as tangential flow filtration (TFF). Typically, TFF requires an effective number of diafiltration volumes to provide a final solution substantially free of the detergent, for example, between about 5 and about 100 diafiltration to achieve a level of the final detergent about 0.01% w/v or less, about 0.005% w/v or less, or about 0.001% w/v or less. Thus, the final solution contains a 100-fold, 200-fold, 300-fold, 400-fold, 500-fold, 600-fold, 700-fold, 800-fold, 800-fold, 1000-fold, or more than 1000-fold less of the detergent compared to the solution prior to the step of removing the detergent from the mixture. The disclosed methods typically yield nanocages after detergent removal by TFF more than 60%, more than 65%, more than 70%, more than 75%, more than 80%, more than 85%, more than 90%, or more than 95%, when measured by saponin content. The resulting nanocage particles are a porous, cage-like, non-liposome, non-micelle particles and preferably, a monodispersion of particles with a diameter ranging between about 30 nm and about 60 nm. Preferably, the step of removing the detergent from the mixture is completed in less than 5 days, less than 3 days, less than 2 days, or less than 1 day. Thus, the method using TFF is generally preferred over dialysis which can take 3-5 days.

Suitable ratios for the lipid, sterol, saponin, and optional additional adjuvant (e.g., TLR4 agonist), components are provided. In some embodiments, the ratio of lipid:additional adjuvant:sterol:saponin mass ratio of 1:1:2:10, or a variation thereof, and the mass ratio of lipid, additional adjuvant, sterol, saponin or any combination thereof is increased or decreased by any value between about 0 and about 3.

Exemplary lipids, additional adjuvants including TLR4 agonists, sterols, and saponins are also provided. The lipid is typically a phospholipid, such as 2-dipalmitoyl-sn-glycero-3-phosphocholine (DPPC). The sterol is most typically cholesterol or a derivative thereof. The saponin can be a natural or synthetic saponin, for example, Quil A or submixture or pure saponin separated therefrom. In some embodiments, the saponin is a natural or synthetic Q-21, or an analog thereof.

Preferred additional adjuvants are TLR4 agonists. An exemplary TLR4 agonist is a lipopolysaccharide (LPS) or a lipid A derivative thereof. In particular embodiments, the lipid A derivative is a monophosphoryl lipid A such as a 4′-monophosporyl lipid A (MPLA) or 3-O-deacylated monophosphoryl lipid A (3D-MPLA).

Other additional adjuvants include, for example, pathogen-associated molecular patterns (PAMPs). In some embodiments, the PAMP is a TLR ligand, a NOD ligand, an RLR ligand, a CLR ligand, an inflammasome inducer, a STING ligand, or a combination thereof. Typically, the additional adjuvant includes a lipid to facilitate incorporation of the adjuvant into the nanocage during self-assemble. Thus, any additional adjuvant, and particularly those that do not already include one, can be modified to include a lipid.

In one embodiment, the lipid is DPPC, the additional adjuvant is a natural or synthetic MPLA, the sterol is cholesterol, and the saponin is Quil A, in a mass ratio of 1:1:2:10. In another embodiment, the lipid is DPPC, the additional adjuvant is a natural or synthetic MPLA, the sterol is cholesterol, and the saponin is Q-21, in a mass ratio of 1:1:2:10.

Nanocage particles prepared by the disclosed methods are provided. Pharmaceutical compositions including a plurality of the nanocage particles, a pharmaceutical carrier, and optionally antigen, are also provided. The composition typically includes an effective amount of the nanocage particles alone or in combination with antigen to increase an immune response in a subject in need thereof. The immune response can be, for example, increasing an antigen-specific antibody response, increasing a response in a germinal center, increasing plasmablast frequency, increasing inflammatory cytokine, increasing drainage of antigen from an injection site, increasing antigen accumulation in a lymph node, increasing permeability of a lymph node, increasing lymph flow, increasing antigen-specific B cell antigen uptake in a lymph nodes, increase a humeral response beyond the proximal lymph node, increase diffusion of antigen into B cell follicles, or a combination thereof.

The particles, particularly those having additional adjuvant, can increase the immune response relative to a control such as the absence of the particles, and/or the presence of another adjuvant such as ADDAVAX™, alum, particles having the same formulation absent the additional adjuvant (e.g., TLR4 agonist), or a liposome or micelle formulation having the same lipid, additional adjuvant (e.g., TLR4 agonist), sterol, and saponin. In some embodiments, the effect of the particles with additional adjuvant is improved compared to another adjuvant such as ISCOMATRIX® or ASO1B.

Methods of using these nanocage particles are also provided. For example, a method of treating a subject in need thereof can include administering the subject a pharmaceutical composition having an effective amount of particles to induce an immune response against an antigen. In some embodiments, the antigen is derived from tumor cells or a microbe, and the subject has or may develop a cancer or infection associated with the tumor cells or microbe. The methods can also include, but need not require, administering the subject an effective amount of the antigen in the same or a separate admixture (e.g., pharmaceutical composition). Preferred methods of administering particles and/or antigen include, but are not limited to, subcutaneous, intramuscular, intradermal, and intravenous injection.

Kits including a plurality of the nanocage particles, for example, in a lyophilized or dried form, or suspended in a pharmaceutically acceptable carrier, are also provided. The kits can also include antigen in a lyophilized or dried form or suspended in a pharmaceutically acceptable carrier. The nanocage particles and antigen can be packaged in a single container or separate containers. Dosage units for administration to a subject in need thereof are also provided.

### BRIEF DESCRIPTION OF THE DRAWINGS

FIGS. 1A-1C are a schematic illustration showing strategy of micelle dilution for ISCOM assembly (FIG. 1A); graph showing DLS volume-based size distribution of MEGA-10 detergent at various concentrations in the presence or absence of ISCOM components (FIG. 1B); and graph showing DLS intensity-weighted size measurements (Z-avg) and polydispersity index (PDI) of 5 mg/mL ISCOMs rapidly diluted from an initial 7.5% (w/v) MEGA-10 to the indicated final concentrations of surfactant (arrows indicate change in size after incubation sample overnight at room temperature) (FIG. 1C).

FIGS. 2A-2D are schematic illustration showing the close-association model of micelle-monomer equilibrium (FIG. 2A); schematic illustration showing TFF system used to derive mass balance equations (FIG. 2B); graph showing concentration (%) over time (increase in diafiltration volumes) from TFF model using a 10 kDa membrane to remove detergent monomers from a mixture with 7.5% MEGA-10 and 5 mg/mL saponin (FIG. 2C); and graph showing concentration (%) over time (increase in diafiltration volumes) from TFF model using a 100 kDa membrane to first concentrate a mixture with 0.15% MEGA-10 and 0.1 mg/mL saponin to 5 mg/mL saponin followed by purification of mixture from detergent monomers (FIG. 2D).

FIGS. 3A-3E are schematic illustration showing the process to generate ISCOMs via dialysis, standard dTFF, and low detergent dTFF (FIG. 3A); graphs showing DLS intensity-based distribution (%) (nm) (FIG. 3B) and DLS number-based distribution (%) (FIG. 3C) over diameters (nm) of final particles including ISCOM prepared from dTFF and dialysis and SMNP prepared from dTFF and dTFF using low detergent; graph showing DLS Z-avg, number average (#-avg) and PDI of these particles (FIG. 3D); and graph showing representative zeta potential distribution of SMNP particles generated via dialysis or dTFF with average and standard deviation provided of three measurements (FIG. 3E).

FIGS. 4A-4C are schematic diagram showing mouse studies immunizing mice with SMNP and HIV antigen (FIG. 4A); ELISA analysis showing absorbance at 450 nm-540 nm of serum IgGs at week 4 after mice immunized with 2 μg of N332-GT2 trimer and 5 μg of SMNP from the different preparations including dialysis, dTFF, and dTFF using low detergent (FIG. 4B); and graph showing serum IgG titers (log₁₀) measured from curves shown in FIG. 4B (FIG. 4C).

FIGS. 5A-5E are chemical structure of the major isomer of QS-21 (FIG. 5A); graphs showing DLS Z-avg (●), vol-avg (◯), number-avg (#), and PDI (□) for attempts to replicate the process used for Quil-A SMNP synthesis with QS-21 using dTFF (FIG. 5B) and low detergent dTFF (FIG. 5C)—error bars omitted for clarity; graphs showing DLS intensity-based and number-based distribution (%) for standard dTFF (FIG. 5D) and low detergent dTFF (FIG. 5E).

FIGS. 6A-6D are graph showing DLS Z-avg size measurements and PDI of 5 mg/mL of QS-21 SMNPs in 7.5% MEGA-10 at various dilutions from 1 to 0.0625% (FIG. 6A); diagram illustrating slow dilution at room temperature to generate SMNPs (FIG. 6B); graph showing DLS Z-avg (●), number-avg (#), and PDI (□) for QS-21 SMNP samples slowly diluted continuously then processed via TFF (dTFF) compared to QS-21 SMNP generated via dialysis (FIG. 6C); and graph showing representative zeta potential distribution of QS-21 SMNPs generated via dTFF or dialysis with average zeta potential of three measurements displayed (FIG. 6D).

FIGS. 7A-7B are graphs showing representative chromatogram of intensity (mV) versus retention time (min) of QS-21 SMNP particles separated on a Jupiter C4 column with acetonitrile gradient ranging from 30 to 95% (FIG. 7A); and representative chromatogram intensity (mV) versus retention time (min) of QS-21 SMNP particles separated on a Accucore C8 column with ispopropanol gradient ranging from 5 to 95% (FIG. 7B).

FIGS. 8A-8B are graphs showing standard curve generated based on changes in MEGA-10 concentration (mg/ml) versus absorbance at 205 nm (AU) (FIG. 8A); standard curve generated based on normalized concentration of MEGA-10 versus diafiltration volumes in permeate from three independent batches of QS-21 SMNPs (FIG. 8B). Dashed lines indicate 95% confidence interval from fitted curves.

FIGS. 9A-9F are graphs showing representative DLS intensity-based distribution (%) versus diameters (nm) (FIG. 9A) and DLS number-based distribution (%) versus diameters (nm) (FIG. 9B) of QS-21 SMNP particles stored at 4° C. or frozen at −20° C. or −80° C. then thawed at room temperature; graphs showing DLS Z-avg (nm) (FIG. 9C) and number-avg (nm) (FIG. 9D) for QS-21 SMNP samples stored at 4° C. or frozen at −20° C. or −80° C. then thawed at room temperature; showing absorbance at 450 nm-540 nm of serum IgGs at week 6 of mice immunized with 2 μg of N332-GT2 trimer and 5 μg of SMNP particles with either QS-21 or QuilA prepared using dialysis or dTFF stored at 4° C. or frozen at −20° C. or −80° C. then thawed at room temperature (FIG. 9E); graph showing serum IgG titers (log₁₀) measured from curves shown in FIG. 9E (FIG. 9F).

FIGS. 10A-10E are schematic diagram showing QS-21 SMNP synthesis at room temperature (RT) through continuous dilution (FIG. 10A); graph showing DLS Z-avg (●), PDI (▪), and number-avg (#) for RT QS-21 SMNP samples from two independent batches (FIG. 10B); absorbance at 450 nm-540 nm of serum IgGs over log (dilution factor) at week two (FIG. 10C) and week four (FIG. 10D) for particles generated via dialysis (♦), dTFF with heating of QS-21 (dTFF-heated ▾) and two independent batches of dTFF SMNPs with QS-21 (dTFF-RT #1 ▪; and dTFF-RT #2 ▴) at room temperature for mice immunized with 2 g of N332-GT2 trimer and 5 μg of SMNP; and graph summarizing of serum IgG titers at week 2 and 4 from (FIG. 10E).

FIGS. 11A-11E are schematic diagram showing staggered room temperature (RT) dilution where the sample is repeatedly partially diluted then let sit until fully equilibrated (FIG. 11A); graph showing DLS Z-avg (◯), PDI (▪), and number-avg (●) for room temperature QS-21 SMNP samples synthesized via staggered dilution compared to FPLC purified SMNPs from dialysis synthesis (FIG. 11B); and graph showing serum IgG titers at week 2 and week 4 from mice immunized with 2 μg of N332-GT2 trimer and 5 μg of SMNP using the staggered dilution room temperature protocol (FIG. 11C); a line graph showing DLS intensity-weighted size (Z-avg) and PDI measured after overnight incubation for fast diluted samples or after an appropriate incubation time for the staggered dilution protocol (FIG. 11D); a bar graph and images showing IFN-γ ELISpot of splenocytes harvested two weeks after immunization and stimulated with a pool of N332-GT2 trimer peptides (FIG. 11E).

### DETAILED DESCRIPTION OF THE INVENTION

### I. Definitions

The terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject.

The term “dosage regime” refers to drug administration regarding formulation, route of administration, drug dose, dosing interval and treatment duration.

The term “effective amount” means the amount or dosage sufficient achieve a therapeutic or non-therapeutic biochemical, pharmacologic, or physiologic effect or result. Are “therapeutically effective amount” means a dosage sufficient to treat, inhibit, or alleviate one or more symptoms of a disease state being treated or to otherwise provide a desired pharmacologic and/or physiologic effect. The precise amount or dosage will vary according to a variety of factors. For example, for therapeutically effective amounts, factors may include subject-dependent variables (e.g., age, immune system health, etc.), the disease, and the treatment being administered. The effect of the effective amount can be relative to a control. Such controls are known in the art and discussed herein, and can be, for example the condition of the subject prior to or in the absence of administration of the drug, or drug combination.

The term “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The term “pharmaceutically acceptable salt”, as used herein, refers to derivatives of the compounds defined herein, wherein the parent compound is modified by making acid or base salts thereof. Lists of suitable salts are found in Remington's Pharmaceutical Sciences, 20th ed., Lippincott Williams & Wilkins, Baltimore, MD, 2000, p. 704; and “Handbook of Pharmaceutical Salts: Properties, Selection, and Use,” P. Heinrich Stahl and Camille G. Wermuth, Eds., Wiley-VCH, Weinheim, 2002.

The terms “inhibit” or “reduce” in the context of inhibition, mean to reduce or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%.

The term “treating” or “preventing” a disease, disorder, or condition includes ameliorating at least one symptom of the disease or condition. Desirable effects of treatment include decreasing the rate of disease progression, ameliorating, or palliating the disease state, and remission or improved prognosis. For example, an individual is successfully “treated” if one or more symptoms associated with cancer are mitigated or eliminated, including, but are not limited to, reducing the proliferation of cancerous cells, decreasing symptoms resulting from the disease, increasing the quality of life of those suffering from the disease, decreasing the dose of other medications required to treat the disease, delaying the progression of the disease, and/or prolonging survival of individuals.

The term “biodegradable”, generally refers to a material that will degrade or erode under physiologic conditions to smaller units or chemical species that are capable of being metabolized, eliminated, or excreted by the subject. The degradation time is a function of composition and morphology.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a compound or method is disclosed and discussed and a number of modifications that can be made to a number compositions, methods, systems, etc. including the compound are discussed, each and every combination and permutation of compound or method and the modifications that are possible are specifically contemplated unless specifically indicated to the contrary. Thus, for example, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. For example, same rationale and corresponding disclose also applies to methods and method steps as is discussed for molecules. Thus, if there are a variety of additional steps that can be performed it is understood that each of these additional steps can be performed with any specific embodiment or combination of embodiments of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

### II. Nanocage Particles

Cage-like nanoparticles composed of saponin, sterol, lipid, and optionally additional adjuvant (e.g., TLR4 agonist) (also referred to as “nanocages” or immune stimulating complexes (ISCOMs)) are provided. A plurality of the nanocages can be used as an adjuvant. In some embodiments, the nanocages further include one or more antigens. In some embodiments, the nanocages do not include or incorporate antigen, but nanocage particles are present in a pharmaceutical composition with antigen (e.g., free antigen). In some embodiments, the nanocage adjuvant and antigen are part of two separate compositions. Exemplary saponins, sterols, lipids, additional adjuvants including TLR4 agonists, and antigens are discussed in more detail below.

Generally, the nanocage adjuvant or ISCOM is formed by mixing the components together in the presence of a detergent in a suitable ratio such that when the detergent is removed, the components self-assemble into nanocages. The size of the nanocages is typically dictated by the properties of the components and the self-assembly process. The disclosed compositions and methods typically yield nanocages in the range of between about 30 nm and about 60 nm, inclusive; or between about 40 nm to about 50 nm, inclusive; with a preferred size being about 40 nm.

The nanocages generally assume a distinctive porous morphology that can be structurally distinguished by transmission electronic microscope (TEM) from lipid monolayer (micelle) and lipid bilayer (liposome) particles. For example, in some embodiments, the morphological structure of the nanocages is the same or similar to the morphological structure of ISCOMATRIX®, as described and imaged in Morelli and Maraskovsky, Chapter 16—ISCOMATRIX Adjuvant in the Development of Prophylactic and Therapeutic Vaccines, Immunopotentiators in Modern Vaccines (Second Edition) 2017, Pages 311-332. Thus, preferably, the particles are not micelles or liposomes.

### A. Saponin

The nanocages typically include one or more saponins. A suitable saponin is one that can induce or enhance an immune response. Saponins from plants have proven to be very effective as adjuvants. Saponins are triterpene and steroid glycosides widely distributed in the plant kingdom. Structurally, saponins are amphiphilic surfactants, which explains their surfactant properties, ability to form colloidal solutions, hemolytic activity, and ability to form mixed micelles with lipids and sterols. The saponins most studied and used as adjuvants are those from Chilean tree Quillaja saponaria, which have cellular and humoral adjuvant activity. Saponins extracts from Quillaja saponaria with adjuvant activity are known and employed in commercial or experimental vaccines formulation.

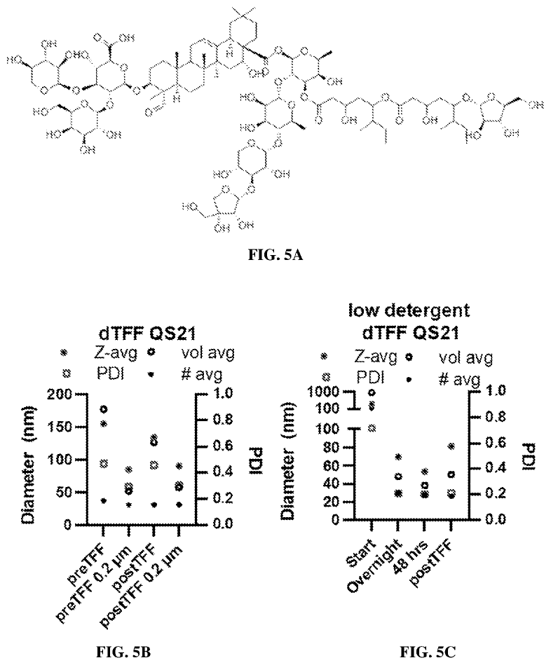

A particular saponin preparation is called Quil A. Quil A is a saponin preparation isolated from the South American tree Ouillaja Saponaria Molina and was first described by Dalsgaard et al. in 1974 (“Saponin adjuvants,” Archiv. fir die gesamte Virus forschung, Vol. 44, Springer Verlag, Berlin, p 243-254) to have adjuvant activity. The isolation of pure saponins or better-defined mixtures from the Quil A product having adjuvant activity and lower toxicity than Quil A have also been described. Purified fragments of Quil A that retain adjuvant activity without the toxicity associated with Quil A (EP 0362 278), for example QS-7 and QS-21 (also known as QA7 and QA21), have been isolated by HPLC. QS-21 is a natural saponin derived from the bark of Quillaja Saponaria Molina, which induces CD8+ cytotoxic T cells (CTLs), Th1 cells and a predominant IgG2a antibody response. QS-21 has been used or is being studied as an adjuvant for various types of vaccines. See also EP 0 362 279 B1 and U.S. Pat. No. 5,057,540. The chemical structure of the major isomer of QS-21 is shown in FIG. 5A.

The isolation and adjuvant activity of other isolated Quil A saponins, including those called QS-17, and QS-18 have also been reported, and can also be used in the disclosed nanocages.

In other embodiments, the saponin is from Quillaja brasiliensis (A. St.-Hil. et Tul.) Mart., which is native to southern Brazil and Uruguay and has saponins that have proven to be effective as adjuvants with a similar activity against viral antigens as Quil A (Silveira et al., Vaccine 29 (2011), 9177-9182).

Other useful saponins are derived from the plants Aesculus hippocastanum or Gyophila struthium. Other saponins which have been described in the literature include escin, which has been described in the Merck index (12th ed: entry 3737) as a mixture of saponins occurring in the seed of the horse chestnut tree, Lat: Aesculus hippocastanum. Its isolation by chromatography and purification (Fiedler, Arzneimittel-Forsch. 4, 213 (1953)), and by ion exchange resins (Erbring et al., U.S. Pat. No. 3,238,190) has been described. Fractions of escin have been purified and shown to be biologically active (Yoshikawa M, et al. (Chem Pharm Bull (Tokyo) August 1996; 44(8): 1454-1464)). Sapoalbin from Gypsophila struthium (R. Vochten et al., 1968, J. Pharm. Belg., 42, 213-226) has also been described.

In other embodiments, the saponin is a synthetic saponin. See, e.g., U.S. Published Application No. 2011/0300177 and U.S. Pat. No. 8,283,456, which describe the Triterpene Saponin Synthesis Technology (TriSST) platform, a convergent synthetic approach in which the four domains in QS-21 (branched trisaccharide+triterpene+linear tetrasaccharide+fatty acyl chain) are synthesized separately and then assembled to produce the target molecule. Each of the domains can be modified independently and then combined to produce a virtually infinite number of rationally designed QS-21 analogs. Initially, fully synthetic QS-21 (SQS-21) was shown to be safe and immunologically active in a Phase 1 clinical trial, and later over 100 analogues were prepared and tested in a systematic sequential series of studies. See, e.g., Ragupathi, et al., Expert Rev Vaccines. 2011 April; 10(4): 463-470. See also Zu, et al., Journal of Carbohydrate Chemistry, Volume 33, 2014—Issue 6, pages 269-97.

In some embodiments, the saponin component is one or more Quil A saponins, or one or more purified fragments of Quil A such as QS-7, QS-17, QS-18, and QS-21. In preferred embodiments, the saponin component is purified QS-21. Typically, the saponin component is in a substantially pure form, for example, at least 70% pure, at least 75% pure, at least 80% pure, at least 85% pure, at least 90% pure, preferably at least 95% pure, and most preferably at least 98% pure.

### B. Sterol

The nanocages typically include one or more sterols. Sterols include β-sitosterol, stigmasterol, ergosterol, ergocalciferol, campesterol, and cholesterol. These sterols are well known in the art, for example cholesterol is disclosed in the Merck Index, 11th Ed., page 341, as a naturally occurring sterol found in animal fat. In preferred embodiments, the sterol is cholesterol or a derivative thereof e.g., ergosterol or cholesteryl hemisuccinate.

### C. Lipid

The nanocages typically include one or more lipids, preferably one or more phospholipids. The lipid can be neutral, anionic, or cationic at physiologic pH. Phospholipids include, but are not limited to, diacylglycerides such as phosphatidic acid (phosphatidate) (PA), phosphatidylethanolamine (cephalin) (PE), phosphatidylcholine (lecithin) (PC), phosphatidylserine (PS), and phosphoinositides, e.g., phosphatidylinositol (PI), phosphatidylinositol phosphate (PIP), phosphatidylinositol bisphosphate (PIP2) and phosphatidylinositol trisphosphate (PIP3), as well as phosphoshingolipids such as ceramide phosphorylcholine (Sphingomyelin) (SPH), ceramide phosphorylethanolamine (Sphingomyelin) (Cer-PE), and ceramide phosphoryllipid, and natural and synthetic phospholipid derivatives such as egg PC (Egg lecithin), egg PG, soy PC, hydrogenated soy PC, sphingomyelin, phosphatidic acid (DMPA, DPPA, DSPA), phosphatidylcholine (DDPC, DLPC, DMPC, DPPC, DSPC, DOPC, POPC, DEPC), phosphatidylglycerol (DMPG, DPPG, DSPG, POPG), phosphatidylethanolamine (DMPE, DPPE, DSPE DOPE), phosphatidylserine (DOPS), and PEG phospholipid (mPEG-phospholipid, polyglycerin-phospholipid, functionalized-phospholipid, terminal activated-phospholipid).

Thus, nanocage can include any one of more of 1,2-Didecanoyl-sn-glycero-3-phosphocholine (DDPC), 1,2-Dierucoyl-sn-glycero-3-phosphate (Sodium Salt) (DEPA-NA), 1,2-Dierucoyl-sn-glycero-3-phosphocholine (DEPC), 1,2-Dierucoyl-sn-glycero-3-phosphoethanolamine (DEPE) 1,2-Dierucoyl-sn-glycero-3[Phospho-rac-(1-glycerol) (Sodium Salt) (DEPG-NA), 1,2-Dilinoleoyl-sn-glycero-3-phosphocholine (DLOPC), 1,2-Dilauroyl-sn-glycero-3-phosphate (Sodium Salt) (DLPA-NA) 1,2-Dilauroyl-sn-glycero-3-phosphocholine (DLPC) 1,2-Dilauroyl-sn-glycero-3-phosphoethanolamine (DLPE), 1,2-Dilauroyl-sn-glycero-3[Phospho-rac-(1-glycerol) (Sodium Salt) (DLPG-NA), 1,2-Dilauroyl-sn-glycero-3[Phospho-rac-(1-glycerol) (Ammonium Salt) (DLPG-NH4), 1,2-Dilauroyl-sn-glycero-3-phosphoserine (Sodium Salt) (DLPS-NA), 1,2-Dimyristoyl-sn-glycero-3-phosphate (Sodium Salt) (DMPA-NA), 1,2-Dimyristoyl-sn-glycero-3-phosphocholine (DMPC), 1,2-Dimyristoyl-sn-glycero-3-phosphoethanolamine (DMPE), 1,2-Dimyristoyl-sn-glycero-3[Phospho-rac-(1-glycerol) (Sodium Salt) (DMPG-NA), 1,2-Dimyristoyl-sn-glycero-3[Phospho-rac-(1-glycerol) (Ammonium Salt) (DMPG-NH4), 1,2-Dimyristoyl-sn-glycero-3[Phospho-rac-(1-glycerol) (Sodium/Ammonium Salt) (DMPG-NH4/NA), 1,2-Dimyristoyl-sn-glycero-3-phosphoserine (Sodium Salt) (DMPS-NA), 1,2-Dioleoyl-sn-glycero-3-phosphate (Sodium Salt) (DOPA-NA), 1,2-Dioleoyl-sn-glycero-3-phosphocholine (DOPC), 1,2-Dioleoyl-sn-glycero-3-phosphoethanolamine (DOPE), 1,2-Dioleoyl-sn-glycero-3[Phospho-rac-(1-glycerol) (Sodium Salt) (DOPG-NA), 1,2-Dioleoyl-sn-glycero-3-phosphoserine (Sodium Salt) (DOPS-NA), 1,2-Dipalmitoyl-sn-glycero-3-phosphate (Sodium Salt) (DPPA-NA), 1,2-Dipalmitoyl-sn-glycero-3-phosphocholine (DPPC), 1,2-Dipalmitoyl-sn-glycero-3-phosphoethanolamine (DPPE), 1,2-Dipalmitoyl-sn-glycero-3[Phospho-rac-(1-glycerol) (Sodium Salt) (DPPG-NA), 1,2-Dipalmitoyl-sn-glycero-3[Phospho-rac-(1-glycerol) (Ammonium Salt) (DPPG-NH4), 1,2-Dipalmitoyl-sn-glycero-3-phosphoserine (Sodium Salt) (DPPS-NA), 1,2-Distearoyl-sn-glycero-3-phosphate (Sodium Salt) (DSPA-NA), 1,2-Distearoyl-sn-glycero-3-phosphocholine (DSPC), 1,2-Distearoyl-sn-glycero-3-phosphoethanolamine (DSPE), 1,2-Distearoyl-sn-glycero-3[Phospho-rac-(1-glycerol) (Sodium Salt) (DSPG-NA), 1,2-Distearoyl-sn-glycero-3[Phospho-rac-(l-glycerol) (Ammonium Salt) (DSPG-NH4), 1,2-Distearoyl-sn-glycero-3-phosphoserine (Sodium Salt) (DSPS-NA), Egg-PC (EPC), Hydrogenated Egg PC (HEPC), Hydrogenated Soy PC (HSPC), 1-Myristoyl-sn-glycero-3-phosphocholine (LYSOPC MYRISTIC), 1-Palmitoyl-sn-glycero-3-phosphocholine (LYSOPC PALMITIC), 1-Stearoyl-sn-glycero-3-phosphocholine (LYSOPC STEARIC), 1-Myristoyl-2-palmitoyl-sn-glycero 3-phosphocholine (Milk Sphingomyelin MPPC), 1-Myristoyl-2-stearoyl-sn-glycero-3-phosphocholine (MSPC), 1-Palmitoyl-2-myristoyl-sn-glycero-3-phosphocholine (PMPC), 1-Palmitoyl-2-oleoyl-sn-glycero-3-phosphocholine (POPC), 1-Palmitoyl-2-oleoyl-sn-glycero-3-phosphoethanolamine (POPE), 1-Palmitoyl-2-oleoyl-sn-glycero-3 [Phospho-rac-(1-glycerol) . . . ](Sodium Salt) (POPG-NA), 1-Palmitoyl-2-stearoyl-sn-glycero-3-phosphocholine (PSPC), 1-Stearoyl-2-myristoyl-sn-glycero-3-phosphocholine (SMPC), 1-Stearoyl-2-oleoyl-sn-glycero-3-phosphocholine (SOPC), and 1-Stearoyl-2-palmitoyl-sn-glycero-3-phosphocholine (SPPC). Any of the lipids can be PEGylated lipids, for example PEG-DSPE.

### D. Adjuvant

The nanocages optionally include one or more adjuvants in addition to a saponin. The additional adjuvant typically has physical and biochemical properties compatible with its incorporation into structure of the nanocage and that do not prevent nanocage self-assembly. The additional adjuvant also typically increases at least one immune response relative to the same nanocage formulation in the absence of the additional adjuvant. Immune responses include, but are not limited to, an increase in an antigen-specific antibody response (e.g., IgG, IgG2a, IgG1, or a combination thereof), an increase in a response in germinal centers (e.g., increase in the frequency of germinal center B cells, an increase in frequencies and/or activation of T follicular helper (Tfh) cells, an increase in B cell presence or residence in dark zone of germinal center or a combination thereof), an increase in plasmablast frequency, an increase in inflammatory cytokine expression (e.g., IL-6, IFN-γ, IFN-α, IL-1β, TNF-α, CXCL10 (IP-10), or a combination thereof), an increase in drainage of antigen from the injection site, an in increase in antigen accumulation in the lymph nodes, an increase in lymph node permeability, an increase in lymph flow, an increase in antigen-specific B cell antigen uptake in lymph nodes, an increase in humoral responses beyond the proximal lymph node, increased diffusion of antigen into B cell follicles, or a combination thereof, when the nanocages are administered to a subject, preferably in combination with an antigen.

### 1. TLR Agonists

In some embodiments, the additional adjuvant is a TLR agonist. In preferred embodiments, the additional adjuvant is a TLR4 agonist. In other embodiments, the additional adjuvant is one or more TLR agonists. TLR4 is a transmembrane protein member of the toll-like receptor family, which belongs to the pattern recognition receptor (PRR) family. Its activation leads to an intracellular signaling pathway NF-κB and inflammatory cytokine production responsible for activating the innate immune system. Classes of TLR agonists include, but are not limited to, viral proteins, polysaccharides, and a variety of endogenous proteins such as low-density lipoprotein, beta-defensins, and heat shock protein.

Exemplary TLR4 agonist include without limitation derivatives of lipopolysaccharides such as monophosphoryl lipid A (MPLA; Ribi ImmunoChem Research, Inc., Hamilton, Mont.) and muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland).

In a preferred embodiment, the TLR4 agonist is a natural or synthetic lipopolysaccharide (LPS), or a lipid A derivative thereof such as MPLA or 3D-MPLA. Lipopolysaccharides are the major surface molecule of, and occur exclusively in, the external leaflet of the outer membrane of gram-negative bacteria. LPS impede destruction of bacteria by serum complements and phagocytic cells and are involved in adherence for colonization. LPS are a group of structurally related complex molecules of approximately 10,000 Daltons in size and contain three covalently linked regions: (i) an O-specific polysaccharide chain (O-antigen) at the outer region (ii) a core oligosaccharide central region (iii) lipid A—the innermost region which serves as the hydrophobic anchor, it includes glucosamine disaccharide units which carry long chain fatty acids.

The biological activities of LPS, such as lethal toxicity, pyrogenicity and adjuvanticity, have been shown to be related to the lipid A moiety. In contrast, immunogenicity is associated with the O-specific polysaccharide component (O-antigen). Both LPS and lipid A have long been known for their strong adjuvant effects, but the high toxicity of these molecules has precluded their use in vaccine formulations. Significant effort has therefore been made towards reducing the toxicity of LPS or lipid A while maintaining their adjuvanticity.

The Salmonella minnesota mutant R595 was isolated in 1966 from a culture of the parent (smooth) strain (Luderitz et al. 1966 Ann. N. Y. Acad. Sci. 133:349-374). The colonies selected were screened for their susceptibility to lysis by a panel of phages, and only those colonies that displayed a narrow range of sensitivity (susceptible to one or two phages only) were selected for further study. This effort led to the isolation of a deep rough mutant strain which is defective in LPS biosynthesis and referred to as S. minnesota R595.

In comparison to other LPS, those produced by the mutant S. minnesota R595 have a relatively simple structure. (i) they contain no O-specific region—a characteristic which is responsible for the shift from the wild type smooth phenotype to the mutant rough phenotype and results in a loss of virulence (ii) the core region is very short—this characteristic increases the strain susceptibility to a variety of chemicals (iii) the lipid A moiety is highly acylated with up to 7 fatty acids.

4′-monophosporyl lipid A (MPLA), which may be obtained by the acid hydrolysis of LPS extracted from a deep rough mutant strain of gram-negative bacteria, retains the adjuvant properties of LPS while demonstrating a toxicity which is reduced by a factor of more than 1000 (as measured by lethal dose in chick embryo eggs) (Johnson et al. 1987 Rev. Infect. Dis. 9 Suppl:S512-S516). LPS is typically refluxed in mineral acid solutions of moderate strength (e.g. 0.1 M HCl) for a period of approximately 30 minutes. This process results in dephosphorylation at the 1 position, and decarbohydration at the 6′ position, yielding MPLA. In some embodiments, the TLR4 agonist is MPLA.

3-O-deacylated monophosphoryl lipid A (3D-MPLA), which can be obtained by mild alkaline hydrolysis of MPLA, has a further reduced toxicity while again maintaining adjuvanticity, see U.S. Pat. No. 4,912,094 (Ribi Immunochemicals). Alkaline hydrolysis is typically performed in organic solvent, such as a mixture of chloroform/methanol, by saturation with an aqueous solution of weak base, such as 0.5 M sodium carbonate at pH 10.5. In some embodiments, the TLR4 agonist is 3D-MPLA.

In some embodiments, the MPLA is a fully synthetic MPLA such as Phosphorylated HexaAcyl Disaccharide (PHAD®), the first fully synthetic monophosphoryl Lipid A available for use as an adjuvant in human vaccines, or Monophosphoryl 3-Deacyl Lipid A (Synthetic) (3D-PHAD®). See also U.S. Pat. No. 9,241,988.

### 2. Other Exemplary Adjuvants

As introduced above, the additional adjuvant typically has physical and biochemical properties compatible with its incorporation into the structure of the nanocage and that do not prevent nanocage self-assembly and increase an immune response. Thus, other suitable adjuvants immunostimulators include those that include a lipid tail or can be modified to contain a lipid tail. Examples of molecules that include a lipid tail, or can be modified to include one, can be, for example, pathogen-associated molecular patterns (PAMPs). PAMPS are recognized by pattern recognition receptors (PRRs). Five families of PRRs have been shown to initiate pro-inflammatory signaling pathways: Toll-like receptors (TLRs), NOD-like receptors (NLRs), RIG-I-like receptors (RLRs), C-type lectin receptors (CLRs) and cytosolic dsDNA sensors (CDSs). Also, some NLRs are involved in the formation of pro-inflammatory complexes called inflammasomes.

Thus, in some embodiments, the adjuvant is a TLR ligand, a NOD ligand, an RLR ligand, a CLR ligand, and inflammasome inducer, a STING ligand, or a combination thereof. Such ligands are known in the art can obtained through commercial vendors such as InvivoGen.

As introduced above, the ligands and other adjuvants can be modified (e.g., through chemical conjugation, for example, maleimide thiol reaction, amine N-hydroxysuccinimide ester reaction, click chemistry, etc.) to include a lipid tail to facilitate incorporation of the adjuvant into the nanocage structure during self-assembly. Preferred lipids will include a 16:0 dipalmitoyl tail such as 1,2-dipalmitoyl-sn-glycero-3-phosphoethanolamine-N-[4-(p-maleimidophenyl)butyramide], these, however, are non-limiting examples. For example, lipids of different lengths are also contemplated. In preferred embodiments, the lipid or lipids is/are unsaturated. Chemically functionalized lipids that that can be used for conjugation are known in the art and commercially available. See, for example, AVANTI® Polar Lipids, Inc. (e.g., “Headgroup Modified Lipids” and “Functionalized Lipids”).

The adjuvant can be an immunostimulatory oligonucleotide, preferable a lipidated immunostimulatory oligonucleotide. Exemplary lapidated immunostimulatory oligonucleotides and methods of making them are described in Liu, et al., Nature Letters, 507:519-22 (+11 pages of extended data) (2014)) (lipo-CpG) and U.S. Pat. No. 9,107,904, that contents of which are incorporated by reference herein in their entireties. In some embodiments, the immunostimulatory oligonucleotide portion of the adjuvant can serve as a ligand for PRRs. Therefore, the oligonucleotide can serve as a ligand for a Toll-like family signaling molecule, such as Toll-Like Receptor 9 (TLR9).

For example, unmethylated CpG sites can be detected by TLR9 on plasmacytoid dendritic cells and B cells in humans (Zaida, et al., Infection and Immunity, 76(5):2123-2129, (2008)). Therefore, the sequence of the oligonucleotide can include one or more unmethylated cytosine-guanine (CG or CpG, used interchangeably) dinucleotide motifs. The ‘p’ refers to the phosphodiester backbone of DNA, as discussed in more detail below, some oligonucleotides including CG can have a modified backbone, for example a phosphorothioate (PS) backbone.

In some embodiments, an immunostimulatory oligonucleotide can contain more than one CG dinucleotide, arranged either contiguously or separated by intervening nucleotide(s). The CpG motif(s) can be in the interior of the oligonucleotide sequence. Numerous nucleotide sequences stimulate TLR9 with variations in the number and location of CG dinucleotide(s), as well as the precise base sequences flanking the CG dimers.

Typically, CG ODNs are classified based on their sequence, secondary structures, and effect on human peripheral blood mononuclear cells (PBMCs). The five classes are Class A (Type D), Class B (Type K), Class C, Class P, and Class S (Vollmer, J & Krieg, A M, Advanced drug delivery reviews 61(3): 195-204 (2009), incorporated herein by reference). CG ODNs can stimulate the production of Type I interferons (e.g., IFNα) and induce the maturation of dendritic cells (DCs). Some classes of ODNs are also strong activators of natural killer (NK) cells through indirect cytokine signaling. Some classes are strong stimulators of human B cell and monocyte maturation (Weiner, GL, PNAS USA 94(20): 10833-7 (1997); Dalpke, A H, Immunology 106(1): 102-12 (2002); Hartmann, G, J of Immun. 164(3):1617-2 (2000), each of which is incorporated herein by reference).

Other PRR Toll-like receptors include TLR3, and TLR7 which may recognize double-stranded RNA, single-stranded and short double-stranded RNAs, respectively, and retinoic acid-inducible gene I (RIG-I)-like receptors, namely RIG-I and melanoma differentiation-associated gene 5 (MDA5), which are best known as RNA-sensing receptors in the cytosol. Therefore, in some embodiments, the oligonucleotide contains a functional ligand for TLR3, TLR7, or RIG-I-like receptors, or combinations thereof.

Examples of immunostimulatory oligonucleotides, and methods of making them are known in the art, see for example, Bodera, P. Recent Pat Inflamm Allergy Drug Discov. 5(1):87-93 (2011), incorporated herein by reference.

In some embodiments, the oligonucleotide includes two or more immunostimulatory sequences.

Microbial cell-wall components such as Pam2CSK4, Pam3CSK4, and flagellin activate TLR2 and TLR5 receptors respectively and can also be used.

### E. Antigen

As discussed herein, antigen refers to the molecule to which an immune response is desired. The antigen can be a component of the nanocage structure itself and/or separate and distinct therefrom (e.g., distinct from the saponin, sterol, lipid, and additional adjuvant (e.g., TLR4 agonist) components). Antigen can also be incorporated into or adsorbed to the nanocage structure. Thus, in some embodiments, the nanocages can optionally include, encapsulate, or incorporate one or more antigens. Such nanocages can thus serve as both adjuvant and antigen in an immunogenic or vaccine formulation. In other embodiments, the nanocages are formed of saponin, sterol, lipid, and optionally additional adjuvant (e.g., additional adjuvant (e.g., TLR4 agonist)) components and absent or free of an antigen. In such embodiments, the nanocages typically serve as an adjuvant only. In some such embodiments, antigen (e.g., free antigen) is present in a pharmaceutical composition in combination with a nanocage adjuvant that is free from/of the antigen.

Antigens can be peptides, proteins, polysaccharides, saccharides, lipids, nucleic acids, or combinations thereof. The antigen can be derived from a virus, bacterium, parasite, plant, protozoan, fungus, tissue or transformed cell such as a cancer or leukemic cell and can be a whole cell or immunogenic component thereof, e.g., cell wall components or molecular components thereof.

Suitable antigens are known in the art and are available from commercial, government, and scientific sources. The antigens are whole inactivated or attenuated organisms, or derived therefrom. These organisms may be infectious organisms, such as viruses, parasites and bacteria. These organisms may also be tumor cells, or derived therefrom. For example, the antigens may be purified or partially purified polypeptides derived from tumors or viral or bacterial sources. The antigens can be recombinant polypeptides produced by expressing DNA or mRNA encoding the polypeptide antigen in a heterologous expression system. The antigens can be DNA or mRNA encoding all or part of an antigenic protein. The DNA may be in the form of vector DNA such as plasmid DNA.

Antigens may be provided as single antigens or may be provided in combination. Antigens may also be provided as complex mixtures of polypeptides or nucleic acids. Exemplary antigens are provided below.

### 1. Viral Antigens

A viral antigen can be isolated from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramnyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue DINS1, Dengue D1NS2, and Dengue D1NS3.

Viral antigens may be derived from a particular strain such as a papilloma virus, a herpes virus, e.g., herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

### 2. Bacterial Antigens

Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamvdia, Chlorobium, Chromatium, Clostridium, Corynebacteriurn, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacteriun, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

### 3. Parasite Antigens

Parasite antigens can be obtained from parasites such as, but not limited to, an antigen derived from Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

### 4. Allergens and Environmental Antigens

The antigen can be an allergen or environmental antigen, such as, but not limited to, an antigen derived from naturally occurring allergens such as pollen allergens (tree-, herb, weed-, and grass pollen allergens), insect allergens (inhalant, saliva, and venom allergens), animal hair and dandruff allergens, and food allergens. Important pollen allergens from trees, grasses and herbs originate from the taxonomic orders of Fagales, Oleales, Pinales and platanaceae including i.a. birch (Betula), alder (Alnus), hazel (Corylus), hornbeam (Carpinus) and olive (Olea), cedar (Cryptomeriaand juniperus), Plane tree (Platanus), the order of Poales including e.g., grasses of the genera Lolium, Phleum, Poa, Cynodon, Dactvlis, Holcus, Phalaris, Secale, and Sorghum, the orders of Asterales and Urticales including i.a. herbs of the genera Ambrosia, Artemisia, and Parietaria. Other allergen antigens that may be used include allergens from house dust mites of the genus Dermatophagoides and Euroglyphus, storage mite e.g Lepidoglyphys, Glycyphagus and Tyrophagus, those from cockroaches, midges and fleas e.g. Blatella, Periplaneta, Chironomus and Ctenocepphalides, those from mammals such as cat, dog and horse, birds, venom allergens including such originating from stinging or biting insects such as those from the taxonomic order of Hymenoptera including bees (superfamily Apidae), wasps (superfamily Vespidea), and ants (superfamily Formicoidae). Still other allergen antigens that may be used include inhalation allergens from fungi such as from the genera Alternaria and Cladosporium.

### 5. Cancer Antigens

A cancer antigen is an antigen that is typically expressed preferentially by cancer cells (i.e., it is expressed at higher levels in cancer cells than on non-cancer cells) and in some instances it is expressed solely by cancer cells. The cancer antigen may be expressed within a cancer cell or on the surface of the cancer cell. The cancer antigen can be MART-1/Melan-A, gp100, adenosine deaminase-binding protein (ADAbp), FAP, cyclophilin b, colorectal associated antigen (CRC)-C017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain, and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-All, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-1, GAGE-2, GAGE-3, GAGE-4, GAGE-5, GAGE-6, GAGE-7, GAGE-8, GAGE-9, BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gpl00Pmel117, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, Imp-1, PlA, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20, or c-erbB-2.

### 6. Tolerogenic Antigens

The antigen can be a tolerogenic antigen. Exemplary antigens are known in the art. See, for example, U.S. Published Application No. 2014/0356384.

In some cases, the tolerogenic antigen is derived from a therapeutic agent protein to which tolerance is desired. Examples are protein drugs in their wild type, e.g., human factor VIII or factor IX, to which patients did not establish central tolerance because they were deficient in those proteins; or nonhuman protein drugs, used in a human. Other examples are protein drugs that are glycosylated in nonhuman forms due to production, or engineered protein drugs, e.g., having non-native sequences that can provoke an unwanted immune response. Examples of tolerogenic antigens that are engineered therapeutic proteins not naturally found in humans including human proteins with engineered mutations, e.g., mutations to improve pharmacological characteristics. Examples of tolerogenic antigens that have nonhuman glycosylation include proteins produced in yeast or insect cells.

Tolerogenic antigens can be from proteins that are administered to humans that are deficient in the protein. Deficient means that the patient receiving the protein does not naturally produce enough of the protein. Moreover, the proteins may be proteins for which a patient is genetically deficient. Such proteins include, for example, antithrombin-III, protein C, factor VIII, factor IX, growth hormone, somatotropin, insulin, pramlintide acetate, mecasermin (IGF-1), β-glucocerebrosidase, alglucosidase-alfa, laronidase (α-L-iduronidase), idursuphase (iduronate-2-sulphatase), galsulphase, agalsidase-beta (α-galactosidase), α-1 proteinase inhibitor, and albumin.

The tolerogenic antigen can be from therapeutic antibodies and antibody-like molecules, including antibody fragments and fusion proteins with antibodies and antibody fragments. These include nonhuman (such as mouse) antibodies, chimeric antibodies, and humanized antibodies. Immune responses to even humanized antibodies have been observed in humans (Getts D R, Getts M T, McCarthy D P, Chastain E M L, & Miller S D (2010), mAbs, 2(6):682-694).

The tolerogenic antigen can be from proteins that are nonhuman. Examples of such proteins include adenosine deaminase, pancreatic lipase, pancreatic amylase, lactase, botulinum toxin type A, botulinum toxin type B, collagenase, hyaluronidase, papain, L-Asparaginase, rasburicase, lepirudin, streptokinase, anistreplase (anisoylated plasminogen streptokinase activator complex), antithymocyte globulin, crotalidae polyvalent immune Fab, digoxin immune serum Fab, L-arginase, and L-methionase.

Tolerogenic antigens include those from human allograft transplantation antigens. Examples of these antigens are the subunits of the various MHC class I and MHC class II haplotype proteins, and single-amino-acid polymorphisms on minor blood group antigens including RhCE, Kell, Kidd, Duffy and Ss.

The tolerogenic antigen can be a self-antigen against which a patient has developed an autoimmune response or may develop an autoimmune response. Examples are proinsulin (diabetes), collagens (rheumatoid arthritis), myelin basic protein (multiple sclerosis). For instance, Type 1 diabetes mellitus (T1D) is an autoimmune disease whereby T cells that recognize islet proteins have broken free of immune regulation and signal the immune system to destroy pancreatic tissue. Numerous protein antigens that are targets of such diabetogenic T cells have been discovered, including insulin, GAD65, chromogranin-A, among others. In the treatment or prevention of T1D, it would be useful to induce antigen-specific immune tolerance towards defined diabetogenic antigens to functionally inactivate or delete the diabetogenic T cell clones.

Tolerance and/or delay of onset or progression of autoimmune diseases may be achieved for various of the many proteins that are human autoimmune proteins, a term referring to various autoimmune diseases wherein the protein or proteins causing the disease are known or can be established by routine testing. In some embodiments, a patient is tested to identify an autoimmune protein and an antigen is created for use in a molecular fusion to create immunotolerance to the protein.

Embodiments can include an antigen, or choosing an antigen from or derived from, one or more of the following proteins. In type 1 diabetes mellitus, several main antigens have been identified: insulin, proinsulin, preproinsulin, glutamic acid decarboxylase-65 (GAD-65), GAD-67, insulinoma-associated protein 2 (IA-2), and insulinoma-associated protein 2 beta (IA-213); other antigens include ICA69, ICA12 (SOX-13), carboxypeptidase H, Imogen 38, GLIMA 38, chromogranin-A, FISP-60, caboxypeptidase E, peripherin, glucose transporter 2, hepatocarcinoma-intestine-pancreas/pancreatic associated protein, S100R, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5. In autoimmune diseases of the thyroid, including Hashimoto's thyroiditis and Graves' disease, main antigens include thyroglobulin (TG), thyroid peroxidase (TPO) and thyrotropin receptor (TSHR); other antigens include sodium iodine symporter (NIS) and megalin. In thyroid-associated ophthalmopathy and dermopathy, in addition to thyroid autoantigens including TSHR, an antigen is insulin-like growth factor 1 receptor. In hypoparathyroidism, a main antigen is calcium sensitive receptor. In Addison's disease, main antigens include 21-hydroxylase, 17α-hydroxylase, and P450 side chain cleavage enzyme (P450scc); other antigens include ACTH receptor, P450c21 and P450c17. In premature ovarian failure, main antigens include FSH receptor and alpha-enolase. In autoimmune hypophysitis, or pituitary autoimmune disease, main antigens include pituitary gland-specific protein factor (PGSF) 1a and 2; another antigen is type 2 iodothyronine deiodinase. In multiple sclerosis, main antigens include myelin basic protein, myelin oligodendrocyte glycoprotein and proteolipid protein. In rheumatoid arthritis, a main antigen is collagen II. In immunogastritis, a main antigen is H+, K+-ATPase. In pernicious angemis, a main antigen is intrinsic factor. In celiac disease, main antigens are tissue transglutaminase and gliadin. In vitiligo, a main antigen is tyrosinase, and tyrosinase related protein 1 and 2. In myasthenia gravis, a main antigen is acetylcholine receptor. In pemphigus vulgaris and variants, main antigens are desmoglein 3, 1 and 4; other antigens include pemphaxin, desmocollins, plakoglobin, perplakin, desmoplakins, and acetylcholine receptor. In bullous pemphigoid, main antigens include BPI80 and BP230; other antigens include plectin and laminin 5. In dermatitis herpetiformis Duhring, main antigens include endomysium and tissue transglutaminase. In epidermolysis bullosa acquisita, a main antigen is collagen VII. In systemic sclerosis, main antigens include matrix metalloproteinase I and 3, the collagen-specific molecular chaperone heat-shock protein 47, fibrillin-1, and PDGF receptor; other antigens include Scl-70, U1 RNP, Th/To, Ku, Jol, NAG-2, centromere proteins, topoisomerase I, nucleolar proteins, RNA polymerase I, II and III, PM-Slc, fibrillarin, and B23. In mixed connective tissue disease, a main antigen is U1snRNP. In Sjogren's syndrome, the main antigens are nuclear antigens SS-A and SS-B; other antigens include fodrin, poly(ADP-ribose) polymerase and topoisomerase. In systemic lupus erythematosus, main antigens include nuclear proteins including SS-A, high mobility group box I (HMGB1), nucleosomes, histone proteins and double-stranded DNA. In Goodpasture's syndrome, main antigens include glomerular basement membrane proteins including collagen IV. In rheumatic heart disease, a main antigen is cardiac myosin. Other autoantigens revealed in autoimmune polyglandular syndrome type 1 include aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and the type 1 interferons interferon alpha, beta, and omega.

In some cases, the tolerogenic antigen is a foreign antigen against which a patient has developed an unwanted immune response. Examples are food antigens. Some embodiments include testing a patient to identify foreign antigen and creating a molecular fusion that comprises the antigen and treating the patient to develop immunotolerance to the antigen or food. Examples of such foods and/or antigens are provided. Examples are from peanut: conarachin (Ara h 1), allergen II (Ara h 2), arachis agglutinin, conglutin (Ara h 6); from apple: 31 kda major allergen/disease resistance protein homolog (Mal d 2), lipid transfer protein precursor (Mal d 3), major allergen Mal d 1.03D (Mal d 1); from milk: .alpha.-lactalbumin (ALA), lactotransferrin; from kiwi: actinidin (Act c 1, Act d 1), phytocystatin, thaumatin-like protein (Act d 2), kiwellin (Act d 5); from mustard: 2S albumin (Sin a 1), 11 S globulin (Sin a 2), lipid transfer protein (Sin a 3), profilin (Sin a 4); from celery: profilin (Api g 4), high molecular weight glycoprotein (Api g 5); from shrimp: Pen a 1 allergen (Pen a 1), allergen Pen m 2 (Pen in 2), tropomyosin fast isoform; from wheat and/or other cereals: high molecular weight glutenin, low molecular weight glutenin, alpha- and gamma-gliadin, hordein, secalin, avenin; from strawberry: major strawberry allergy Fra a 1-E (Fra a 1), from banana: profilin (Mus xp 1).

Many protein drugs that are used in human and veterinary medicine induce immune responses, which create risks for the patient and limits the efficacy of the drug. This can occur with human proteins that have been engineered, with human proteins used in patients with congenital deficiencies in production of that protein, and with nonhuman proteins. It would be advantageous to tolerize a recipient to these protein drugs prior to initial administration, and it would be advantageous to tolerize a recipient to these protein drugs after initial administration and development of immune response. In patients with autoimmunity, the self-antigen(s) to which autoimmunity is developed are known. In these cases, it would be advantageous to tolerize subjects at risk prior to development of autoimmunity, and it would be advantageous to tolerize subjects at the time of or after development of biomolecular indicators of incipient autoimmunity. For example, in Type 1 diabetes mellitus, immunological indicators of autoimmunity are present before broad destruction of beta cells in the pancreas and onset of clinical disease involved in glucose homeostasis. It would be advantageous to tolerize a subject after detection of these immunological indicators prior to onset of clinical disease.

### 7. Neoantigens and Personalized Medicine

In some embodiments the antigen is a neoantigen or a patient-specific antigen. Recent technological improvements have made it possible to identify the immune response to patient-specific neoantigens that arise as a consequence of tumor-specific mutations, and emerging data indicate that recognition of such neoantigens is a major factor in the activity of clinical immunotherapies (Schumacher and Schreidber, Science, 348(6230):69-74 (2015). Neoantigen load provides an avenue to selectively enhance T cell reactivity against this class of antigens.

Traditionally, cancer vaccines have targeted tumor-associated antigens (TAAs) which can be expressed not only on tumor cells but in the normal tissues (Ito, et al., Cancer Neoantigens: A Promising Source of Immunogens for Cancer Immunotherapy. J Clin Cell Immunol, 6:322 (2015) doi:10.4172/2155-9899.1000322). TAAs include cancer-testis antigens and differentiation antigens, and even though self-antigens have the benefit of being useful for diverse patients, expanded T cells with the high-affinity TCR (T-cell receptor) needed to overcome the central and peripheral tolerance of the host, which would impair anti-tumor T-cell activities and increase risks of autoimmune reactions.

Thus, in some embodiments, the antigen is recognized as “non-self” by the host immune system, and preferably can bypass central tolerance in the thymus. Examples include pathogen-associated antigens, mutated growth factor receptor, mutated K-ras, or idiotype-derived antigens. Somatic mutations in tumor genes, which usually accumulate tens to hundreds of fold during neoplastic transformation, could occur in protein-coding regions. Whether missense or frameshift, every mutation has the potential to generate tumor-specific antigens. These mutant antigens can be referred to as “cancer neoantigens” Ito, et al., Cancer Neoantigens: A Promising Source of Immunogens for Cancer Immunotherapy. J Clin Cell Immunol, 6:322 (2015) doi:10.4172/2155-9899.1000322. Neoantigen-based cancer vaccines have the potential to induce more robust and specific anti-tumor T-cell responses compared with conventional shared-antigen-targeted vaccines. Recent developments in genomics and bioinformatics, including massively parallel sequencing (MPS) and epitope prediction algorithms, have provided a major breakthrough in identifying and selecting neoantigens.

Methods of identifying, selecting, and validating neoantigens are known in the art. See, for example, Ito, et al., Cancer Neoantigens: A Promising Source of Immunogens for Cancer Immunotherapy. J Clin Cell Immunol, 6:322 (2015) doi:10.4172/2155-9899.1000322, which is specifically incorporated by reference herein in its entirety. For example, as discussed in Ito, et al., a non-limiting example of identifying a neoantigen can include screening, selection, and optionally validation of candidate immunogens. First, the whole genome/exome sequence profile is screened to identify tumor-specific somatic mutations (cancer neoantigens) by MPS of tumor and normal tissues, respectively. Second, computational algorithms are used for predicting the affinity of the mutation-derived peptides with the patient's own HLA and/or TCR. The mutation-derived peptides can serve as antigens for the compositions and methods disclosed herein. Third, synthetic mutated peptides and wild-type peptides can be used to validate the immunogenicity and specificity of the identified antigens by in vitro T-cell assay or in vivo immunization.

### III. Methods of Making Nanocages

Methods of making nanocage adjuvant particles composed of saponin, sterol, lipid, and optionally additional adjuvant (e.g., TLR4 agonist) are provided.

Methods generally include a step of (a) mixing one or more saponins, one or more lipids, one or more sterols, optionally one or more additional adjuvants (e.g., TLR4 agonist), and optionally one or more antigens in the presence of a detergent, and (b) removing detergent. As the detergent is removed, the components self-assemble into a dispersion of nanocages. Typically, the dispersion is a monodispersion. In some embodiments, the monodispersion is of particles of approximately 40 nm.

In some embodiments, the mixing step is optionally followed by an incubation step, a dilution and/or concentration step, and a filtration or dialysis step.

Once the detergent is removed the remaining nanocage solution can be sterile filtered using, for example, a 0.2 μm filter.

The nanocages can be purified, for example from loose components such as free additional adjuvant (e.g., TLR4 agonist), by chromatography, for example Fast Protein Liquid Chromatograph (FPLC). Suitable columns include Sephacryl S-500 HR or a similar SEC column.

Preferably, few or no liposomes or micelles are formed. However, certain preparations may yield a small fraction of worm-like micelles with a main fraction containing cage-like particles. If liposomes and/or micelles are formed during the preparation, the nanocages can be selected or separated from the liposomes and/or micelles, for example during purification.

Size/morphology can be measured by dynamic light scattering (DLS) and negative-stain TEM can be used to compare batch-to-batch homogeneity.

Antigen can be added in the presence of detergent. In such embodiments, antigen is present in solution with the other components of the nanocages, and can be incorporated into the structure of nanocages during self-assembly, when detergent is removed.

In some embodiments, antigen is not included in solution with the other components of the nanocages in the presence detergent. Antigen can be added after the detergent is removed, and thus after self-assembly is complete. In such embodiments, it is believed that the antigen will remain free and untethered or unincorporated in the nanocage.

### A. Mixing and Solubilization

In some embodiments, the methods include a step of solubilize or mixing one or more components of lipids, cholesterol saponin, and optionally additional adjuvant in a high concentration of a detergent or surfactant (e.g., MEGA-10). In some embodiments, one or more of the components is in an aqueous stock solution preferably including detergent and the stock solutions prior to mixing.

Preferably, the detergent is a non-ionic detergent and mixing occurs when the detergent concentration is above its critical micelle concentration (CMC). An exemplary non-ionic detergent is Decanoyl-N-methylglucamide (MEGA-10). In some embodiments, the non-ionic detergent is about 20% w/v of the stock solution. In other embodiments, a low concentration of non-ionic detergent is used to facilitate downstream process of detergent removal, for example about 3%-10% w/v, preferably 7.5% w/v.

Other suitable detergents include surfactants with high (>~1 mM) critical micelle concentration (CMC) to minimize required dilution to assemble nanocage structures. These detergents include sodium cholate (CMC~14 mM), sucrose monocholate (CMC~4.7 mM), MEGA-9 (CMC~25 mM), MEGA-8, nonylthiomaltoside, (CMC~2.4 mM), decylmatoside (CMC~1.8 mM), octylthioglucoside (CMC-9 mM), heptylthioglucoside (CMC~30 mM), octylglucoside (CMC~25 mM), deoxyBICHAP (CMC~1.4 mM), BIGCHAP (CMC~2.9 mM), CHAPSO (CMC~8 mM), CHAPS (CMC~8 mM). While detergents with lower CMC may be used, these would increase buffer required for dilution, leading to increased processing time.

Temperature can be adjusted to facilitate solubilization of the detergent or other components of the solution. In some embodiments, the solution(s) is maintained at room temperature during preparation. In other embodiments, the solution(s) is heated above room temperature, e.g., 37° C., 50-60° C., or 60-70° C. during preparation. In preferred embodiments, the solution(s) is kept at a temperature for enhanced solubilization with minimal degradation of any of the components in the mixture. In some embodiments, the solution of each component is kept at a different temperature for promoting solubilization prior to mixing, e.g., heating of DPPC and cholesterol at 60° C. and MPLA at 37° C. to facilitate solubilization into MEGA-10 micelles.

In some cases, the sterol is cholesterol, the saponin is QS-21, and the lipid is 1,2-Dipalmitoyl-sn-glycero-3-phosphocholine (DPPC), optionally in further combination with MPLA as the additional adjuvant.

The components are mixed in a ratio suitable to form nanocages when the detergent is removed.

In a particular embodiments, the components of an ISCOM have a mass ratio of 1:1:5 of lipid:cholesterol:saponin, for example 1:1:5 of saponin:cholesterol:DPPC.

In another particular embodiment, where the ISCOM includes an additional adjuvant (i.e., Saponin-MPLA NanoParticles (SMNP)), the components have a mass ratio is 1:1:2:10 of Lipid:additional adjuvant (e.g., TLR4 agonist):Sterol:Saponin, for example, 1:1:2:10 of DPPC:MPLA:cholesterol:Saponin.

In some embodiments, the nanocage includes Lipid:Sterol:Saponin with a mass ratio of 5:3:2.

The mass ratio of any component or combination thereof can be increased or decreased by any value between about 0 and about 10.

In some embodiments, the components are mixed in the following sequence: sterol, lipid, optionally additional adjuvant (e.g., TLR4 agonist), and saponin.

Inclusion of the Toll-like receptor 4 agonist monophosphoryl lipid A (MPLA) in ISCOM particles yields a promising next-generation adjuvant termed Saponin-MPLA NanoParticles (SMNP). In some embodiments, the nanocages include cholesterol as the sterol, DPPC as the lipid, MPLA as the TLR4 agonist, and Quil-A as the saponin. In one embodiment, the nanocages include a mass ratio 1:1:2:10-DPPC:MPLA:Cholesterol:Quil-A. In another embodiment, the nanocages include a mass ratio 1:1:2:10-DPPC:MPLA:Cholesterol:QS-21.

Generally, the solution from the mixing step is allowed to equilibrate. In some embodiments, the incubation step after the mixing step is immediately followed by a dilution step.

### B. Controlling Nanocage Self-Assembly from Detergent Micelles

The disclosed methods typically next include a step of controllably dissociating detergent micelles into monomers such that nanocages can self-assemble. Such methods typically include manipulating the micelle-monomer equilibrium.

The methods can include reducing the concentration of detergent to achieve a desired concentration of the detergent.

In some embodiments, the methods include dilution of the mixture to achieve a desired concentration of the detergent, increasing ionic strength of the mixture, changing the temperature of the solution, adding one or more detergent-depleting agents to mixture, or a combination thereof.

### 1. Dilution

In some embodiments, the method includes a step of dilution of the mixture prior to detergent removal. As shown in the Examples, in the case of process a solution of Quil-A-based ISCOM components (Quil-A saponin, cholesterol, and 1,2-dipalmitoyl-sn-glycero-3-phosphocholine [DPPC]) in MEGA-10 micelles through a 10 kDa MWCO hollow fiber TFF filter filtration was ineffective with high operating pressures and low permeate fluxes due to the high concentration of material. Thus, in some cases, the solution after mixing one or more components of lipids, cholesterol, and saponin in a high concentration of a detergent is diluted to achieve optimal operating pressures and/or higher permeate fluxes to achieve successful purification of ISCOM in a shorter period of time.

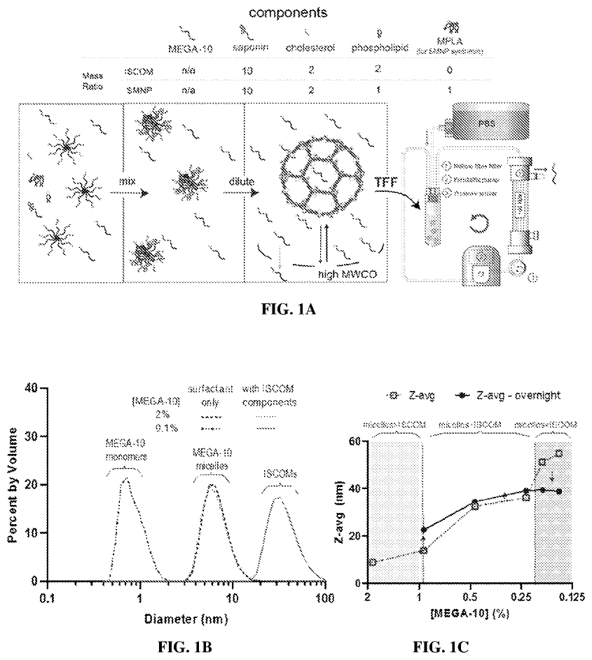

In some embodiments, the mixture is diluted below the critical micelle concentration (CMC) of the detergent. The CMC of a detergent is the concentration of a detergent in which micelles start to form. As shown in the Examples and in FIGS. 1B-1C, three regions of solution behavior were identified as a function of detergent final concentration: (1) at high MEGA-10 concentrations (>1%), micelles dominated with sizes of ~10 nm that did not change after overnight incubation; (2) at intermediate MEGA-10 concentrations (1%-0.2%), both micelles and ISCOMs were present, and DLS measured intermediate sizes between micelles and ISCOMs (10-40 nm), with minor increases in particle size after overnight incubation indicating the formation of ISCOMs; and (3) at low MEGA-10 concentrations (<0.2%), the low micelle content due to proximity to the detergent CMC led to formation of larger species (~60 nm) that tended to condense into the expected ~40 nm diameter size of ISCOMs after overnight incubation.

Thus, in some embodiments, the solution is diluted prior to the step of detergent removal, for example by 5-fold, 10-fold, 20-fold, 30-fold, 40-fold, 50-fold, 60-fold, 70-fold, 80-fold, 90-fold, 100-fold, or more than 100-fold. In preferred embodiments, the mixture solution is diluted to an effective concentration to provide the maximal yield of ISCOMs, minimize detergent micelles, provide optimal operating pressures, and/or allow sufficient permeate fluxes.

In other embodiments, the mixture is diluted to about or just above CMC, prior to the step of detergent removal; for example, in the case of MEGA-10, to a concentration between about 0.2% and 1%, inclusive. In preferred embodiments, the mixture is diluted below CMC to generate ISCOMs and/or to minimize detergent micelles, prior to the step of detergent removal. In the case of MEGA-10, the CMC of MEGA-10 in H₂O is about ~6-7 mM (0.21%). Thus, in some embodiments, where MEGA-10 is used as the detergent, the mixture is diluted to a concentration of MEGA-10 of less than 0.21%, less than 0.2%, less than 0.19%, less than 0.18%, less than 0.17%, less than 0.16%, less than 0.15%, less than 0.14%, less than 0.13%, less than 0.12%, less than 0.11%, less than 0.10%, less than 0.09%, less than 0.08%, less than 0.07%, less than 0.06%, or less than 0.05%.

### Dilution Rate

In some embodiments, dilution is achieved rapidly, for example, in less than 30 minutes, less than 20 minutes, less than 10 minutes, or less than 5 minutes, or less than one minute. In other embodiments, dilution is achieved slowly over a period of between 1 hour and about 24 hours, preferably between about 5 hours to 20 hours, more preferably between about 10 hours to 15 hours.

In some embodiments, dilution is achieved continuously (continuous dilution), with buffer being added dropwise to the solution until a desired dilution is achieved, for example over a period of between 1 hour and about 24 hours, preferably between about 5 hours to 20 hours, more preferably between about 10 hours to 15 hours. In continuous dilution, the solution may not fully reach equilibrium during its dilution as buffer was constantly added. In one embodiment, dilution is carried out overnight by adding buffer dropwise to the solution at a rate to achieve 10-fold dilution per hour for 10 hours.

In other embodiments, dilution is achieved stepwise (staggered discontinuous dilution), i.e., allowing solution to equilibrate prior to the next dilution step. For example, staggered dilution can be performed by diluting in 10-fold steps relative to the initial volume and allowing samples sufficient time to equilibrate e.g., 30 minutes, before performing the next dilution step. In further embodiments, dilution is achieved by a combination of continuous and stepwise dilution steps.

In the case of a more hydrophobic fragment of Quil-A such as QS-21, a slow dilution method (e.g., continuous dilution or staggered discontinuous dilution) is preferable over fast dilution for promoting the formation of homogenous nanocage particles (e.g., ISCOM, SMNP). In one embodiment, dilution is performed at a rate of 10-fold per hour, to gradually move from the starting solution into the “low micelle concentration” region (0.075%), allowing the ISCOM components to reach their equilibrium assembly state before getting to their final structures as the MEGA-10 concentration dropped.

### 2. Additional and Alternative Means

Although a dilution step is a preferred method to control the disassembly of detergent micelles into monomers, other means of controlling the detergent micelle-monomer equilibrium can also be used in alternative or addition to dilution. For example, addition or removal of salts from a solution can alter the CMC of ionic detergents (generally increasing ionic strength decreases CMC). Thus, the detergent mixture with nanocage components could be generated with a high ionic strength then the salts could be gradually removed through methods known in the art such as ultrafiltration, precipitation or via chromatographic columns. In some embodiments, ionic strength of the solution is increased to promote self-assembly of the one or more components of lipids, cholesterol, saponin and optionally an additional adjuvant into a dispersion of nanocage particles.

Other means of removing detergents can be employed by addition of surfactant depleting agents such as fumed silica, activated charcoal, or other commercially available detergent-binding resins (e.g., SDR HYPERD®, HIPPR™ Detergent Removal Resin). Similar to dilution, through controlled addition of these detergent-depleting agents, micelles would gradually dissociate into monomers, allowing self-assembly of nanocage particles. In some embodiments, the concentration of detergent(s) is reduced, for example, via one or more surfactant depleting agents, to promote self-assembly of the one or more components of lipids, cholesterol, saponin and optionally an additional adjuvant into a dispersion of nanocage particles.

In some embodiments, two or more of these strategies are combined to control the detergent micelle-monomer equilibrium, for example, increase in ionic strength of the starting material followed by the dilution which could be done with a lower ionic strength solution.

### C. Incubation

In some embodiments, the method includes one or more incubation steps. The incubation step allows the solution from the mixing step to equilibrate, for example, for between about 0.1 and about 100 hours, inclusive; preferably between about 2 hours and about 24 hours, inclusive; at about room temperature and optionally protected from light. In preferred embodiments, the solution is allowed to equilibrate overnight.

In preferred embodiments, the mixture is incubated for an effective amount of time for the maximal formation of nanocages. In some embodiments, the incubation step is carried out after a rapid dilution step. In one embodiment, the mixture is rapidly diluted to final desirable concentration followed by an overnight incubation to promote the formation of nanocages.

### D. Detergent Removal

Generally, the detergent is removed from the mixture solution. In some cases, the solution is also concentrated and/or buffer exchanged for the final buffer solution such as phosphate buffered saline or a buffer system suitable for cryopreservation. In some embodiments, detergent is removed using dialysis, centrifugation, filtration (e.g., tangential flow filtration (TFF), ultrafiltration), or combinations thereof. For example, international publication No. WO 2001/076625A1 describes ISCOM coupled to a polypeptide purified using ultracentrifugation; Sjolander A et al., Vaccine 19 (2001)2661-2665 describes ISCOM by ultrafiltration.

In some embodiments, the method involves one or more steps of dialysis. However, as shown in the Examples, ISCOMs prepared by dialysis may contain a small proportion of worm-like species compared to those prepared by TFF. Thus, in preferred embodiments, the method does not involve dialysis, or does not involve dialysis alone for detergent removal.

In TFF, a sample solution flows through the feed channel and along (tangent to) the surface of the membrane as well as through the membrane. The crossflow prevents buildup of molecules at the surface that can cause fouling. Thus, the TFF process prevents the rapid decline in flux rate seen in direct flow filtration allowing a greater volume to be processed per unit area of membrane surface. TFF is a linearly scalable size-based separations technique that facilitates GMP production of ISCOMs at clinically relevant scales. Thus, in preferred embodiments, a scalable and facile process for manufacturing of ISCOMs and SMNP involve TFF. TFF has been used in preparation of other nanoparticles such as described in US Published Application No. 2019/0382539A1.

In some embodiments, the detergent is removed via one or more steps of TFF. In preferred embodiments, detergent removal via one or more steps of TFF results in minimal ring-like micelles and worm-like micelles primarily composed of saponin and sterol compared to detergent removal via dialysis alone. Thus, in further preferred embodiments, detergent removal via one or more steps of TFF improves the robustness of nanocage (e.g., ISCOM or SMNP) production. Preferably, the yield after detergent removal by TFF is more than 60%, more than 65%, more than 70%, more than 75%, more than 80%, more than 85%, more than 90%, or more than 95%, when measured by saponin content.

In some embodiments, the solution is diafiltered with suitable buffer by TFF using filtration membranes with a molecular weight cutoff (MWCO) between about 10 kDa and about 300 kDa, inclusive; preferably between about 30 kDa and about 150 kDa, inclusive; and more preferably between about 50 kDa and about 100 kDa, inclusive. In one embodiment, hollow fiber filtration membranes with a 100 kDa pore size cutoff are used in the TFF. In preferred embodiments, filtration membranes with MWCO that is effective to be used with TFF for optimal operating pressures and permeate fluxes. In one embodiment, a 100 kDa pore size membrane provides about 10-fold higher permeate flexes relative to a 10 kDa pore size membrane.

In some embodiments, the mixture solution requires between about 5 and about 100 diafiltration volumes (i.e., total permeate volume relative to sample volume), preferably between about 10 and about 70 diafiltration volumes, more preferably between about 20 and about 50 diafiltration volumes, to be substantially free of any small molecular weight impurity such as the detergent from the mixture. In one embodiment, the diafiltration volume is about 10. In some embodiments, the reduction in any small molecular weight impurity such as the detergent is about 100-fold, 200-fold, 300-fold, 400-fold, 500-fold, 600-fold, 700-fold, 800-fold, 800-fold, 1000-fold, or more than 1000-fold, preferably achieved in less than 100 diafiltration volumes, or less than 60 diafiltration volumes, or less than 50 diafiltration volumes, or less than 40 diafiltration volumes, or less than 30 diafiltration volumes, or less than 20 diafiltration volumes, or less than 10 diafiltration volumes.

In some embodiments, the micellar solution is first diluted to below its CMC followed by a concentration step and constant volume diafiltration by TFF, preferably on a high MWCO membrane (e.g., 100 kDa). In one embodiment, the solution is diluted to about 50-fold followed by about 40-50 diafiltration volumes for concentrating the saponin and a further 5-6 diafiltration volumes for purification.

In preferred embodiments, the final solution after TFF is substantially free of the detergent from the mixture, for example, to a level about 0.01% w/v or less, about 0.005% w/v or less, about 0.004% w/v or less, about 0.003% w/v or less, about 0.002% w/v or less, or about 0.001% w/v or less of the detergent. In one embodiment, where MEGA-10 is used as the detergent, the concentration of MEGA-10 is about 0.001% w/v or less after the step of detergent removal by TFF.

### E. Storage

Methods of preparing nanocage particles for short-term and long-term storage are also described. In some embodiments, the disclosed nanocage particles or the formulations thereof are stored at room temperature, or between about 4° C. and about 20° C. In other embodiments, the disclosed nanocage particles or the formulations thereof are subject to freezing for long-term storage between about −1° C. and about −80° C., preferably between about −10° C. and about −30° C., or about −20° C. In preferred embodiments, the method of preparing nanocage particles for storage yields a good recovery after the desired period of time, with minimal loss of its biological adjuvant properties.

In some cases, the ice crystallization induces a mechanical stress which leads to destabilization of nanocage particles, so cryoprotectants can be added to the formulation prior freezing to protect and further stabilize nanoparticles. Thus, in some embodiments, the nanocage formulation further includes one or more cryoprotectants. Examples of cryoprotectants include sucrose, trehalose, raffinose, stachyose, verbascose, mannitol, glucose, lactose, maltose, maltotriose-heptaose, dextran, hydroxyethyl starch, sorbitol, glycerol, arginine, histidine, lysine, proline, dimethylsulfoxide, or any combination thereof.

The concentration of the cryoprotectant in the formulation is in an effective amount to yield a good rate of recovery after one or more freezing-thawing cycles, for example, the concentration of the cryoprotectant ranges from about 0.05% to about 50% w/v (e.g., from about 0.05% to about 25% w/v, from about 1% to 15% w/v, from about 3% to about 12.5% w/v, from about 1% to about 8% w/v, or from about 2% to about 7% w/v).

After one or more freezing-thawing cycles, nanocage particles may increase in size. Preferably the percentage of the nanocage particles with increase average size of particles is about 30% or less, about 20% or less, about 15% or less, about 10% or less, about 5% or less, after storage at −20° C. or lower for at least one month, e.g., as measured dynamic light scattering (DLS).

Generally, the method provides a good recovery rate after one or more freezing-thawing cycles after a period of time (e.g., one month or one year), about 50% or more, 60% or more, 70% or more, 80% or more, 90% or more, 95% or more, or 99% or more, as compared to the number of nanocage particles prior to after one or more freezing-thawing cycles. In preferred embodiments, after thawing from being frozen and stored with or without one or more cryoprotectants for a period of time the nanocage particles retain their size, cage-like morphology, sample homogeneity, and adjuvant activity.

Freeze-drying is a commonly used method to stabilize nanoparticles. Thus, in some embodiments, the formulations are freeze dried, with or without a cryoprotectant.

### F. Exemplary Preferred Embodiments

Various aspects of the disclosed compositions and methods can be varied as discussed throughout disclosure.

The Examples below establish particularly preferred embodiments. See, e.g., Table 4. For example, in exemplary embodiments the appearance of the mixture is a colorless to whitish, clear to slightly opalescent suspension; the mass ration of saponin:sterol:phospholipid:additional adjuvant, optionally QS-21:cholesterol:DPPC:MPLA, is 10:2±0.3:1±0.3:1±0.3; the particle size (Z_{avg}) is 40 nm-80 nm; the polydispersity index (PDI) is ≤0.25; residual detergent, optionally MEGA-10, is ≤1 μg/mL; and the particle morphology is cage-like e.g., as characterized via negative stain TEM; or any combination thereof.

### IV. Formulations

### A. Pharmaceutical Compositions

Pharmaceutical compositions including nanocage adjuvants, antigens, and the combination thereof are provided. Pharmaceutical compositions can be for administration by parenteral (intramuscular, intraperitoneal, intravenous (IV), intradermal, or subcutaneous injection), or transmucosal (nasal, vaginal, rectal, or sublingual) routes of administration or using bioerodible inserts and can be formulated in dosage forms appropriate for each route of administration.

In some embodiments, the compositions are administered systemically, for example, by intravenous or intraperitoneal administration, in an amount effective for delivery of the compositions to targeted cells.

Most typically, the compositions are administered by intramuscular, intradermal, subcutaneous, or intravenous injection or infusion, or by intranasal delivery.

In certain embodiments, the compositions are administered locally, for example by injection directly into a site to be treated. In some embodiments, the compositions are injected or otherwise administered directly to one or more tumors. Typically, local injection causes an increased localized concentration of the compositions which is greater than that which can be achieved by systemic administration.

In some embodiments, the compositions are delivered by using a catheter or syringe. Other means of delivering such compositions include using infusion pumps (for example, from Alza Corporation, Palo Alto, Calif.) or incorporating the compositions into polymeric implants (see, for example, P. Johnson and J. G. Lloyd-Jones, eds., Drug Delivery Systems (Chichester, England: Ellis Horwood Ltd., 1987), which can effect a sustained release of the composition to the immediate area of the implant.

As further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired.

An exemplary dosage range for antigen and adjuvant components of a vaccine are about 10 μg to about 500 μg of antigen and about 10 μg to about 1000 μg of adjuvant. In some embodiments, the dosage range of the antigen is between about 10 ng and about 500 μg, or about 10 ng and 100 μg.

Adjuvant dosages can also be determined based on activity or units. For example, concentration of the nanocages (e.g., saponin-MPLA) can be quantified by measuring the sterol (e.g., cholesterol) content of the purified products (sigma MAK043). The sterol (e.g., cholesterol) quantification is then referred to as units of activity. In some embodiments, this value is further multiplied by the mass ratio of saponin:sterol (e.g., QuilA:cholesterol) to get an estimated saponin (e.g., Quil-A) content. In some embodiments, the unit dosage of a nanocage adjuvant is between about 1 U and about 10 U, or between about 2 U and about 7 U, or between about 2.5 U and about 5 U.

### 1. Formulations for Parenteral Administration

In a preferred embodiment the nanocage adjuvant, the antigen, or a combination thereof are administered in an aqueous solution, by parenteral injection.

The formulation can be in the form of a suspension or emulsion. In general, pharmaceutical compositions are provided including an effective amount of the adjuvant and optionally include pharmaceutically acceptable diluents, preservatives, solubilizers, emulsifiers, adjuvants and/or carriers. Such compositions can include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate.

The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

### 2. Formulations for Topical and Mucosal Administration

The adjuvants and/or antigens can be applied topically. Topical administration can include application to the lungs (pulmonary), nasal, oral (sublingual, buccal), vaginal, or rectal mucosa.

Compositions can be delivered to the lungs while inhaling and traverse across the lung epithelial lining to the blood stream when delivered either as an aerosol or spray dried particles having an aerodynamic diameter of less than about 5 microns.

A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent® nebulizer (Mallinckrodt Inc., St. Louis, Mo.); the Acorn® II nebulizer (Marquest Medical Products, Englewood, Colo.); the Ventolin® metered dose inhaler (Glaxo Inc., Research Triangle Park, N.C.); and the Spinhaler® powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa will typically be spray dried drug particles, which may be incorporated into a tablet, gel, capsule, suspension or emulsion. Standard pharmaceutical excipients are available from any formulator.

### B. Immunogenic Compositions

The adjuvants disclosed herein can be used in immunogenic compositions and as components in vaccines. Typically, immunogenic compositions disclosed herein include an adjuvant, an antigen, or a combination thereof. When administered to a subject in combination, the adjuvant and antigen can be administered in separate pharmaceutical compositions, or they can be administered together in the same pharmaceutical composition.

When present in the same pharmaceutical composition, or administered in combination, an adjuvant and an antigen can be referred to as a vaccine.

### V. Methods of Use

Methods of using the disclosed compositions including a nanocage adjuvant alone or in combination with an antigen can be administered in an effective amount to induce, increase, or enhance an immune response. Immune response typically refers to responses that induce, increase, or perpetuate the activation or efficiency of innate or adaptive immunity.

The compositions can also be used to promote tolerance, e.g., to an allergen or autoimmune antigen.

The composition can be delivered parenterally (e.g., by subcutaneous, intradermal, or intramuscular injection) through the lymphatics, or by systemic administration through the circulatory system (e.g., by intravenous injection or infusion). In some embodiments, a nanocage adjuvant and an antigen are administered in the same manner or route. In other embodiments, the different compositions are administered in two or more different manners or routes.

In some embodiments, the compositions are delivered non-systemically. In some embodiments, at least the adjuvant alone or in combination with antigen is delivered locally. In some embodiments, the compositions are delivered by subcutaneous injection. In some embodiments, the composition is administered at a site adjacent to or leading to one or more lymph nodes which are close to the site in need of an immune response (i.e., close to a tumor or site of infection). In some embodiments, the composition is injected into the muscle. In some embodiments, the composition is administered in multiple doses at various locations throughout the body. The composition can also be administered directly to a site in need of an immune response (e.g., a tumor or site of infection).

In some embodiments, particularly those for the treatment of cancer and some infections, the nanocage adjuvant is administered without administering an antigen. It is believed that the nanocage adjuvant can still increase immune response to, for example endogenous tumor antigens or microbial antigens, without administering any further antigens to the subject.

### A. Methods of Increasing an Immune Response

The immune response can be induced, increased, or enhanced by the composition compared to a control. In some embodiments, a nanocage adjuvant including an additional adjuvant such as a TLR4 agonist is administered to a subject in need thereof in an effective amount to increase an antigen-specific antibody response (e.g., IgG, IgG2a, IgG1, or a combination thereof), increase a response in germinal centers (e.g., increase the frequency of germinal center B cells, increase frequencies and/or activation T follicular helper (Tfh) cells, increase B cell presence or residence in dark zone of germinal center or a combination thereof), increase plasmablast frequency, increase inflammatory cytokine expression (e.g., IL-6, IFN-γ, IFN-α, IL-10, TNF-α, CXCL10 (IP-10), or a combination thereof), increase drainage of antigen from the injection site, increase antigen accumulation in the lymph nodes, increase lymph node permeability, increase lymph flow, increase antigen-specific B cell antigen uptake in lymph nodes, increase a humeral response beyond the proximal lymph node, increase diffusion of antigen into B cell follicles, or a combination thereof.

The control can be, for example, no adjuvant or another adjuvant. Thus, in some embodiments, the disclosed nanocage adjuvants including an additional adjuvant such as a TLR4 agonist that can increase an immune response in a subject relative to, for example, Addavax, Alum, ISCOMATRIX®, ASO1B, or another adjuvant.

The disclosed nanocage adjuvants can be used, for example, to induce an immune response, when administering the antigen alone or in combination with an alternative adjuvant is ineffectual. In some embodiments, the nanocage adjuvant may reduce the dosage of adjuvant, antigen, or both required to induce, increase, or enhance an immune response; or reduce the time needed for the immune system to respond following administration.

Nanocage adjuvants may be administered as part of prophylactic vaccines or immunogenic compositions which confer resistance in a subject to subsequent exposure to infectious agents, or as part of therapeutic vaccines, which can be used to initiate or enhance a subject's immune response to a pre-existing antigen, such as a viral antigen in a subject infected with a virus or with cancer.

The desired outcome of a prophylactic or therapeutic immune response may vary according to the disease or condition to be treated, or according to principles well known in the art. For example, an immune response against an infectious agent may completely prevent colonization and replication of an infectious agent, affecting “sterile immunity” and the absence of any disease symptoms. However, a vaccine against infectious agents may be considered effective if it reduces the number, severity or duration of symptoms; if it reduces the number of individuals in a population with symptoms; or reduces the transmission of an infectious agent. Similarly, immune responses against cancer, allergens or infectious agents may completely treat a disease, may alleviate symptoms, or may be one facet in an overall therapeutic intervention against a disease.

### B. Tolerance

The compositions and methods disclosed herein may also be used to promote tolerance. Tolerogenic therapy aims to induce immune tolerance where there is pathological or undesirable activation of the normal immune response. Such embodiments may also include co-administration of an immunosuppressive agent such as rapamycin.

Tolerogenic vaccines deliver antigens with the purpose of suppressing immune responses (e.g., induce or increase a suppressive immune response) and promoting robust long-term antigen-specific immune tolerance. For example, Incomplete Freund's Adjuvant (IFA) mixed with antigenic peptides stimulates Treg proliferation (and/or accumulation) and IFA/Insulin peptide prevents type I diabetes onset in susceptible mice, though this approach is ineffective in reversing early onset type I diabetes (Fousteri, G., et al., 53:1958-1970 (2010)). The compositions and methods disclosed herein are also useful for controlling the immune response to an antigen. For example, in some embodiments, the compositions are used as part of a tolerizing vaccine.

An exemplary composition typically contains an antigen, or a nucleic acid encoding an antigen as in DNA vaccines, and a nanocage adjuvant. The antigen, for example, a self-antigen, depends on the disease to be treated, and can be determined by one of skill in the art. Exemplary self-antigens and other tolerizing antigens are discussed in more detail above. Adjuvant and antigen can be administered in an amount effective to, for example, increase immunosuppression.

### C. Diseases to Be Treated

### 1. Infectious Diseases

The compositions are useful for treating acute or chronic infectious diseases. Thus, the compositions can be administered for the treatment of local or systemic viral infections, including, but not limited to, immunodeficiency (e.g., HIV), papilloma (e.g., HPV), herpes (e.g., HSV), encephalitis, influenza (e.g., human influenza virus A), and common cold (e.g., human rhinovirus) viral infections. For example, pharmaceutical formulations including the composition can be administered topically to treat viral skin diseases such as herpes lesions or shingles, or genital warts. The composition can also be administered to treat systemic viral diseases, including, but not limited to, AIDS, influenza, the common cold, or encephalitis.

Representative infections that can be treated, include but are not limited to infections cause by microorganisms including, but not limited to, Actinomyces, Anabaena, Bacillus, Bacteroides, Bdellovibrio, Bordetella, Borrelia, Campylobacter, Caulobacter, Chlamnydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Histoplasma, Hyphomicrobium, Legionella, Leishmania, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, Yersinia, Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Plasmodium vivax, Trypanosoma brucei, Entamoeba histolytica, Toxoplasma gondii, Trichomonas vaginalis and Schistosoma mansoni.

In some embodiments, the type of disease to be treated or prevented is a chronic infectious disease caused by a bacterium, virus, protozoan, helminth, or other microbial pathogen that enters intracellularly.

In particular embodiments, infections to be treated are chronic infections cause by a hepatitis virus, a human immunodeficiency virus (HIV), a human T-lymphotrophic virus (HTLV), a herpes virus, an Epstein-Barr virus, or a human papilloma virus.

### 2. Cancer

The compositions may be used for treating cancer, by for example, stimulating or enhancing an immune response in host against the cancer. The types of cancer that may be treated with the provided compositions and methods include, but are not limited to, the following: bladder, brain, breast, cervical, colo-rectal, esophageal, kidney, liver, lung, nasopharangeal, pancreatic, prostate, skin, stomach, uterine, ovarian, testicular and hematologic.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The compositions can be administered as an immunogenic composition or as part of vaccine, such as prophylactic vaccines, or therapeutic vaccines, which can be used to initiate or enhance a subject's immune response to a pre-existing antigen, such as a tumor antigen in a subject with cancer.

The desired outcome of a prophylactic or therapeutic immune response may vary according to the disease, according to principles well known in the art. Similarly, immune responses against cancer, may alleviate symptoms, or may be one facet in an overall therapeutic intervention against a disease. For example, administration of the composition may reduce tumor size, or slow tumor growth compared to a control. The stimulation of an immune response against a cancer may be coupled with surgical, chemotherapeutic, radiologic, hormonal and other immunologic approaches in order to affect treatment.

### 3. Subjects in Need of Tolerance

The compositions that increase tolerance disclosed herein can be used to inhibit immune-mediated tissue destruction for example in a setting of inflammatory responses, autoimmune and allergic diseases, and transplant rejection.

a. Inflammatory and Autoimmune Disorders

In certain embodiments, the disclosed compositions are used to treat an inflammatory response or autoimmune disorder in a subject. For example, the disclosed methods can be used to prophylactically or therapeutically inhibit, reduce, alleviate, or permanently reverse one or more symptoms of an inflammatory response or autoimmune disorder. An inflammatory response or autoimmune disorder can be inhibited or reduced in a subject by administering to the subject an effective amount of a composition in vivo, or cells modulated by the composition ex vivo.

Representative inflammatory responses and autoimmune diseases that can be inhibited or treated include, but are not limited to, rheumatoid arthritis, systemic lupus erythematosus, alopecia areata, ankylosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Bechet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia—fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

b. Transplant Rejection

In another embodiment, the disclosed compositions and methods can be used prophylactically or therapeutically to reduce or inhibit graft rejection or graft verse host disease. Transplant rejection occurs when a transplanted organ or tissue is not accepted by the body of the transplant recipient. Typically rejection occurs because the immune system of the recipient attacks the transplanted organ or tissue. The disclosed methods can be used to promote immune tolerance of the transplant or graft by the receipt by administering to the subject an effective amount of a composition in vivo, or cells modulated by the composition ex vivo.

i. Transplants

The transplanted material can be cells, tissues, organs, limbs, digits or a portion of the body, for example the human body. The transplants are typically allogenic or xenogenic. The disclosed compositions are administered to a subject in an effective amount to reduce or inhibit transplant rejection. The compositions can be administered systemically or locally by any acceptable route of administration. In some embodiments, the compositions are administered to a site of transplantation prior to, at the time of, or following transplantation. In one embodiment, compositions are administered to a site of transplantation parenterally, such as by subcutaneous injection.

In other embodiments, the compositions are administered directly to cells, tissue or organ to be transplanted ex vivo. In one embodiment, the transplant material is contacted with the compositions prior to transplantation, after transplantation, or both.

In other embodiments, the compositions are administered to immune tissues or organs, such as lymph nodes or the spleen.

The transplant material can also be treated with enzymes or other materials that remove cell surface proteins, carbohydrates, or lipids that are known or suspected of being involved with immune responses such as transplant rejection.

### (a) Cells

Populations of any types of cells can be transplanted into a subject. The cells can be homogenous or heterogenous. Heterogeneous means the cell population contains more than one type of cell. Exemplary cells include progenitor cells such as stem cells and pluripotent cells which can be harvested from a donor and transplanted into a subject. The cells are optionally treated prior to transplantation as mentioned above.

### (b) Tissues

Any tissue can be used as a transplant. Exemplary tissues include skin, adipose tissue, cardiovascular tissue such as veins, arteries, capillaries, valves; neural tissue, bone marrow, pulmonary tissue, ocular tissue such as corneas and lens, cartilage, bone, and mucosal tissue. The tissue can be modified as discussed above.

### (c) Organs

Exemplary organs that can be used for transplant include, but are not limited to kidney, liver, heart, spleen, bladder, lung, stomach, eye, tongue, pancreas, intestine, etc. The organ to be transplanted can also be modified prior to transplantation as discussed above.

One embodiment provides a method of inhibiting or reducing chronic transplant rejection in a subject by administering an effective amount of the composition to inhibit or reduce chronic transplant rejection relative to a control.

ii. Graft-Versus-Host Disease (GVHD)

The disclosed compositions and methods can be used to treat graft-versus-host disease (GVHD) by administering an effective amount of the composition to alleviate one or more symptoms associated with GVHD. GVHD is a major complication associated with allogeneic hematopoietic stem cell transplantation in which functional immune cells in the transplanted marrow recognize the recipient as “foreign” and mount an immunologic attack. It can also take place in a blood transfusion under certain circumstances. Symptoms of GVD include skin rash or change in skin color or texture, diarrhea, nausea, abnormal liver function, yellowing of the skin, increased susceptibility to infection, dry, irritated eyes, and sensitive or dry mouth.

In another embodiment, the disclosed compositions and methods for inducing or perpetuating a suppressive immune response can be used prophylactically or therapeutically to suppress allergies and/or asthma and/or inflammation. Allergies and/or asthma and/or inflammation can be suppressed, inhibited or reduced in a subject by administering to the subject an effective amount of a composition that promotes an immune suppressive immune response or tolerance as described above.

### D. Combination Therapies

In some embodiments, the compositions are administered in further combination with one or more additional therapeutic agents. The agents can be administered in the same or separate pharmaceutical composition from the adjuvant, antigen, or combination thereof.

In some embodiments, the compositions are administered in combination with a conventional therapeutic agent used for treatment of the disease or condition being treated. Conventional therapeutics agents are known in the art and can be determined by one of skill in the art based on the disease or disorder to be treated. For example, if the disease or condition is cancer, the compositions can be co-administered with a chemotherapeutic drug; or if the disease or condition is a bacterial infection, the compositions can be co-administered with an antibiotic.

When administered as a cancer vaccine, the disclosed compositions may be administered in combination with a checkpoint inhibitor (PD1, CTLA4, TIM3, etc.).

### E. Treatment Regimens

The nanocage adjuvants alone or more typically in combination with an antigen can be administered as a vaccine that includes a first (“prime”) and optionally one or more (“boost”) administrations. Thus, in some embodiments, a vaccine is administered 2, 3, 4, or more times, for example, 1, 2, 3, 4, 5, 6, 7, 8, 9, 10, 11, 12, 13, 14, or 15 days, weeks, months, or years apart.

### VI. Kits

Dosage units and stocks of lyophilized nanocage adjuvant alone or in combination with antigen, as well as dosage units and stocks of nanocage adjuvant alone or in combination with antigen in a pharmaceutically acceptable carrier for shipping and storage and/or administration are also provided, and can form part of a kit, for example a vaccination kit.

Components of the kit may be packaged individually and can be sterile. In some embodiments, a pharmaceutically acceptable carrier containing an effective amount of nanocage adjuvant alone or in combination with antigen is shipped and stored in a sterile vial. The sterile vial may contain enough nanocage adjuvant alone or in combination with antigen for one or more doses. In some embodiments, the adjuvant and antigen are in separate containers, which may be combined before administration.

In some embodiments, the adjuvant, the antigen, or both are in a dried or lyophilized form. The kit may include a pharmaceutical carrier that can be used to resuspend the adjuvant, antigen, or combination thereof. In some embodiments, the kit includes adjuvant and/or antigen, or a combination of adjuvant and antigen already suspended in a pharmaceutical carrier.

Nanocage adjuvant alone or in combination with antigen may be shipped and stored in an amount or volume suitable for administration, or may be provided in a concentrated form that is diluted prior to administration. In another embodiment, a pharmaceutically acceptable carrier containing an effective amount of nanocage adjuvant alone or in combination with antigen can be shipped and stored in a syringe. Kits can also contain syringes of various capacities or vessels with deformable sides (e.g., plastic vessels or plastic-sided vessels) that can be squeezed to force a liquid composition out of an orifice. The size and design of the syringe will depend on the route of administration. For example, in one embodiment, a syringe for administering the compositions locally, may be capable of accurately delivering a smaller volume. Larger syringes, pumps and/or catheters can also be provided. Any of the kits can include instructions for use.

The disclosed invention can be further understood by the following numbered paragraphs.
1. A method of making nanocage particles including the steps of
(a) solubilizing and/or mixing one or more components of lipids, cholesterol, saponin and optionally an additional adjuvant, in the presence of a detergent to form detergent micelles,
(b) controllably dissociating the detergent micelles into monomers; and
(c) removing the detergent from the mixture,
wherein the step of (b) and/or (c) promotes self-assembly of the one or more components of lipids, cholesterol, saponin and optionally an additional adjuvant into a dispersion of nanocage particles.
2. The method of paragraph 1, wherein step (b) includes reducing the concentration of detergent to achieve a desired concentration of the detergent.
3. A method of making nanocage particles including the steps of
(a) solubilizing and/or mixing one or more components of lipids, cholesterol, saponin and optionally an additional adjuvant, in the presence of a detergent to form detergent micelles,
(b) reducing the concentration of detergent to achieve a desired concentration of the detergent; and
(c) removing the detergent from the mixture,
wherein the step of (b) and/or (c) promotes self-assembly of the one or more components of lipids, cholesterol, saponin and optionally an additional adjuvant into a dispersion of nanocage particles.
4. The method of any one of paragraphs 1-3, wherein step (b) includes a step of dilution of the mixture of (a) to achieve a desired concentration of the detergent before the step (c).
5. The method of paragraph 4, wherein the dilution step is a rapid dilution step achieving a desired concentration of the detergent in less than 30 minutes, less than 20 minutes, less than 10 minutes, or less than 5 minutes, or less than one minute.
6. The method of paragraph 4, wherein the dilution step is a continuous dilution step achieving a desired concentration of the detergent adding buffer dropwise to the solution over a period of between about 1 hour and about 24 hours.
7. The method of paragraph 6, wherein the continuous dilution step is carried out at a rate to achieve 10-fold dilution per hour for 10 hours.
8. The method of paragraph 6, wherein the dilution step is a staggered discontinuous dilution step achieving a desired concentration of the detergent via stepwise addition of buffer, wherein after each step of adding buffer, the mixture is allowed to equilibrate prior to the next step of adding buffer,
optionally the staggered discontinuous dilution step is performed diluting in 10-fold steps relative to the initial volume and allowing samples sufficient time to equilibrate mixture, before performing the next dilution step.
9. The method of any one of paragraphs 1-8, wherein step (b) includes increasing the ionic strength of the mixture, changing the temperature, adding one or more detergent-depleting agents to change the detergent micelle-monomer equilibrium.
10. The method of any one of paragraphs 2-9, wherein the desired concentration of the detergent is about or below the critical micelle concentration (CMC) of the detergent.
11. The method of any one of paragraphs 1-10, wherein the method further includes an incubation step after step (a) and/or after the dilution step, prior to before (c).
12. The method of paragraph 11, wherein the incubation step is for an effective amount of time for the formation of nanocages.
13. The method of paragraph 12, wherein the incubation step is between about 2 hours and about 24 hours.
14. The method of any one of paragraphs 1-13, wherein the step of removing the detergent from the mixture is carried out via dialysis, centrifugation, filtration, or combinations thereof.
15. The method of paragraph 14, wherein the step of removing the detergent from the mixture is carried out using a filtration membrane with a molecular weight cutoff (MWCO) between about 10 kDa and about 100 kDa, inclusive.
16. The method of paragraph 14, wherein the filtration is tangential flow filtration (TFF),
optionally wherein the TFF requires an effective number of diafiltration volumes to provide a final solution substantially free of the detergent,
optionally, wherein the effective number of diafiltration volumes is between about 5 and about 100 diafiltration.
17. The method of paragraph 16, wherein the final solution is substantially free of the detergent when the detergent is at a level about 0.01% w/v or less, about 0.005% w/v or less, or about 0.001% w/v or less.
18. The method of any one of paragraphs 14-17, wherein the final solution contains a 100-fold, 200-fold, 300-fold, 400-fold, 500-fold, 600-fold, 700-fold, 800-fold, 800-fold, 1000-fold, or more than 1000-fold less of the detergent compared to the solution prior to the step of removing the detergent from the mixture.
19. The method of any one of paragraphs 14-18, wherein the yield of nanocages after detergent removal by TFF is more than 60%, more than 65%, more than 70%, more than 75%, more than 80%, more than 85%, more than 90%, or more than 95%, when measured by saponin content.
20. The method of any one of paragraphs 14-19, wherein the step of removing the detergent from the mixture is completed in less than 5 days, less than 3 days less than 2 days, or less than 1 day.
21. The method of any one of paragraphs 1-20, wherein (i) the mass ratio of lipid:additional adjuvant:sterol:saponin is 1:1:2:10, (ii) the mass ratio of lipid:sterol:saponin is 1:1:5, (iii) or a variation thereof wherein the mass ratio of lipid, additional adjuvant, sterol, saponin or any combination thereof is increased or decreased by any value between about 0 and about 3.
22. The method of any one of paragraphs 1-21, wherein the nanocage particles are a porous, cage-like nanoparticles.
23. The method of any one of paragraphs 1-22, wherein the nanocage particles are a monodispersion of particles with a diameter ranging between about 30 nm and about 60 nm.
24. The method of any one of paragraphs 1-23, wherein the nanocage particles are non-liposome, non-micelle particles.
25. The method of any one of paragraphs 1-24, wherein the lipid is phospholipid.
26. The method of any one of paragraphs 1-25, wherein the lipid is 2-Dipalmitoyl-sn-glycero-3-phosphocholine (DPPC).
27. The method of any one of paragraphs 1-26, wherein the sterol is cholesterol or a derivative thereof.
28. The method of any one of paragraphs 1-27, including the additional adjuvant, wherein the additional adjuvant is a TLR4 agonist.
29. The method of paragraph 28, wherein the TLR4 agonist is a lipopolysaccharide (LPS) or a lipid A derivative thereof.
30. The method of paragraph 28 or 29, wherein the TLR4 agonist is a natural or synthetic monophosphoryl lipid A (MPLA) or a derivative thereof.
31. The method of paragraph 30, wherein the MPLA or derivative thereof is natural or synthetic 4′-monophosporyl lipid A (MPLA) or 3-O-deacylated monophosphoryl lipid A (3D-MPLA).
32. The method of any one of paragraphs 1-27, including the additional adjuvant, wherein the additional adjuvant is a pathogen-associated molecular pattern (PAMP).
33. The method of paragraph 32, wherein the PAMP includes a lipid.
34. The method of paragraph 31 or 32, wherein the PAMP is a TLR ligand, a NOD ligand, an RLR ligand, a CLR ligand, an inflammasome inducer, a STING ligand, or a combination thereof.
35. The method of any one of paragraphs 1-34, wherein the saponin is a natural or synthetic saponin.
36. The method of any one of paragraphs 1-35, wherein the mass ration of saponin:sterol:phospholipid:additional adjuvant is 10:2±0.3:1±0.3:1±0.3
37. The method of any one of paragraphs 1-36, wherein the saponin is Quil A or submixture or pure saponin separated therefrom.
38. The method of any one of paragraphs 1-37, wherein the saponin a natural or synthetic Q-21, or an analog thereof.
39. The method of any one of paragraphs 1-38, wherein the mixture is a colorless to whitish, clear to slightly opalescent suspension; the particle size (Z_{avg}) is 40 nm-80 nm; the polydispersity index (PDI) is ≤0.25; residual detergent, optionally MEGA-10, is ≤1 μg/mL; and the particle morphology is cage-like or any combination thereof.
40. The method of paragraph 39, wherein the mixture is a colorless to whitish, clear to slightly opalescent suspension; the particle size (Z_{avg}) is 40 nm-80 nm; the polydispersity index (PDI) is ≤0.25; residual detergent, optionally MEGA-10, is ≤1 g/mL; and the particle morphology is cage-like.
41. The method of any one of paragraphs 1-40, wherein the lipid is DPPC, the additional adjuvant is a natural or synthetic MPLA, the sterol is cholesterol, and the saponin is Quil A.
42. The method of paragraph 41, wherein the DPPC:MPLA:cholesterol:Quil A are in a mass ratio of 1:1:2:10.
43. The method of any one of paragraphs 1-40, wherein the lipid is DPPC, the additional adjuvant is a natural or synthetic MPLA, the sterol is cholesterol, and the saponin is QS-21.
44. The method of paragraph 43, wherein the mass ration of QS-21:cholesterol:DPPC:MPLA is 10:2±0.3:1±0.3:1±0.3
45. The method of paragraph 43, wherein the DPPC:MPLA:cholesterol:Q-21 are in a mass ratio of 1:1:2:10.
46. The method of paragraph 44, wherein the mixture is a colorless to whitish, clear to slightly opalescent suspension; the particle size (Z_{avg}) is 40 nm-80 nm; the polydispersity index (PDI) is ≤0.25; residual detergent, optionally MEGA-10, is ≤1 g/mL; and the particle morphology is cage-like or any combination thereof.
47. The method of paragraph 44, wherein the mixture is a colorless to whitish, clear to slightly opalescent suspension; the particle size (Z_{avg}) is 40 nm-80 nm; the polydispersity index (PDI) is ≤0.25; residual detergent, residual MEGA-10, is ≤1 g/mL; and the particle morphology is cage-like.
48. A nanocage particle made according to the method of any one of paragraphs 1-47.
49. A pharmaceutical composition including a plurality of the nanocage particles of paragraph 48 and a pharmaceutical carrier.
50. The pharmaceutical composition of paragraph 49, including an effective amount of the nanocage particles to increase an immune response in a subject in need thereof.
51. The pharmaceutical composition of paragraph 50, wherein the immune response is selected from the group consisting of increasing an antigen-specific antibody response, increasing a response in a germinal center, increasing plasmablast frequency, increasing inflammatory cytokine, increasing drainage of antigen from an injection site, increasing antigen accumulation in a lymph node, increasing permeability of a lymph node, increasing lymph flow, increasing antigen-specific B cell antigen uptake in a lymph nodes, or a combination thereof.
52. The pharmaceutical composition of paragraph 50 or 51, wherein the nanocage particles increase the immune response relative to a control.
53. The pharmaceutical composition of paragraph 52, wherein the control is the absence of particles.
54. The pharmaceutical composition of paragraph 52, wherein the control is nanocage particles having the same formulation absent the additional adjuvant.
55. The pharmaceutical composition of paragraph 52, wherein the control is a liposome or micelle including the same lipid, additional adjuvant, sterol, and saponin.
56. The pharmaceutical composition of any one of paragraphs 49-55 further including an antigen to which an immune response is desired.
57. A method of treating a subject in need thereof including administering the subject the pharmaceutical composition of any one of paragraphs 49-56 in an effective amount to induce an immune response against an antigen.
58. The method of paragraph 57, wherein the antigen is derived from tumor cells or a microbe.
59. The method of paragraph 57, wherein the subject has or may develop a cancer or infection associated with tumor cells or microbe.
60. The method of paragraphs 57-59 further including administering the subject an effective amount of the antigen.
61. The method of paragraph 60, wherein the antigen is in the same or a separate pharmaceutical composition from the particles.
62. The method of any one of paragraphs 57-61, wherein the particles alone or in combination with the antigen are administered to the subject by subcutaneous, intramuscular, intradermal, or intravenous injection.
63. A kit including a plurality of the nanocage particles made according to the method of any one of paragraphs 1-47 in a lyophilized or dried form, or suspended in a pharmaceutically acceptable carrier.
64. The kit of paragraph 63, further including antigen in a lyophilized or dried form, or suspended in a pharmaceutically acceptable carrier.
65. The kit of paragraphs 63 or 64, wherein the particles and antigen are in a single container or separate containers.

The present invention will be further understood by reference to the following non-limiting examples.

### EXAMPLES

### Example 1: Manufacturing of Next-Generation Immune Stimulating Complexes by Controlled Lipid Self-Assembly

### Material and Methods

Generation of saponin and detergent/lipid mixture: ISCOM and SMNP adjuvants was prepared as previously described (M. Silva, et al., Sci. Immunol. 2021, 6). Briefly, solutions of cholesterol (Avanti Polar Lipids Cat #700000) and DPPC (Avanti Polar Lipids Cat #850355) were prepared in Milli-Q water containing 20% w/vol MEGA-10 (Sigma D6277) detergent at 60° C. Quil-A saponin (InvivoGen; vac-quil) was dissolved in either Milli-Q water or 20% w/vol MEGA-10 to compare processing to QS-21 (Desert King) which was soluble in 20% MEGA-10. Saponin solutions were solubilized at 37° C. expect for the room temperature QS-21 preparations. For ISCOMs, all components were mixed at mass ratio of 5:1:1 (saponin:chol:DPPC) at 60° C. followed by dilution with pre-heated PBS to a final concentration of 5 mg/ml saponin and 7.5% MEGA-10. SMNP was formulated in a similar manner but included MPLA (Avanti Polar Lipids 699800P) at a mass ratio of 10:2:1:1 (saponin:chol:DPPC:MPLA). MPLA stock solutions were generated in 20% MEGA-10 at 10 mg/mL by with heating at 37° C. ISCOMs and SMNP concentrations are reported in terms of the amount of saponin. For in vivo studies, saponin content was calculated by measuring the concentration of cholesterol (Cholesterol Quantitation kit; Millipore Sigma; Cat #MAK043) in the preparation and assuming all lipids incorporated in proportion to their relative amounts included in the synthesis. Prior to removing detergent through dialysis or TFF, the mixture was allowed to equilibrate at room temperature for at least two hours. Dilution for Quil-A SMNPs was performed in less than one minute to reach 0.15% MEGA-10. For “low detergent” synthesis of Quil-A SMNP, the initial lipid mixture was prepared with 5% MEGA-10 at a 1:1.5 mass ratio of MEGA-10 to saponin. For overnight QS-21 SMNP dilution, buffer was dropwise added to the sample at a rate to achieve 10×/hr for 10 hours. Staggered dilution was performed by diluting in 10-fold steps relative to the initial volume and allowing samples to equilibrate for 30 minutes before performing the next dilution step.

ISCOM/SMNP self-assembly via dialysis: The saponin and lipid mixture was placed in 10 kDa molecular weight cut-off (MWCO) dialysis membranes (ThermoFisher, Slide-A-Lyzer) and extensively dialyzed against PBS for five days at 25° C. with buffer changes at least once a day. The adjuvant solution was passed through an 0.2 μm sterile filter, and concentrated using 50K MWCO Amicron Ultra (Sigma) spin filters. Quil-A preparations were further purified by FPLC using a Sephacryl S-500 HR size exclusion column.

ISCOM/SMNP self-assembly via TFF: The saponin and lipid mixtures were diluted with PBS at room temperature (20-25° C.) until stable ISCOM formation was observed (expect for low detergent Quil-A SMNP in which dilution was done at 60° C.). For QS-21 SMNP, the dilution rate was controlled to occur at a rate of 10-fold per hour. Samples were then placed on a KrosFlo KR2i tangential flow filtration system (Repligen) using 20 cm² 100 kDa MWCO mPES membranes. QS-21 and Quil-A batch sizes ranged from 1 to 15 mg of QS-21 using the 20 cm² membrane. The “low detergent” Quil-A batch size was increased to 50 mg of Quil-A. For processing, the samples were concentrated to approximately 1 mg/mL prior to having MEGA-10 removed by performing 10 diafiltration volumes against PBS. Concentration of MEGA-10 in the permeate was estimated based on its absorbance at 205 nm with an empirically determined mass extinction coefficient of 20 mg⁻¹cm⁻¹ (FIG. 8A). After TFF, Quil-A ISCOM and SMNP preparations were further purified by FPLC using a Sephacryl 5-500 HR size exclusion column.

Characterization of particle preparations: Dynamic light scattering (DLS) and zeta potential measurements were made on a Zetasizer Nano ZSP (Malvern). Nanoparticle micrographs were acquired using Transmission Electron Microscopy (TEM) on a JEOL 2100F microscope (200 kV) or on a FEI Tecnai (120 kV). The microscopes were with a magnification range of 10,000-60,000×.

Modeling of TFF with detergent micelles. The surfactant monomer-micelle equilibrium was represented by the closed-association model in which n detergent monomers associate into one detergent micelle as shown in Equation 1 (I. A. Nyrkova, A. N. Semenov, Eur. Phys. J. E 2005, 17, 327).

$$nM ↔ {M}_{n} \qquad ( 1 )$$

Where M is the concentration of surfactant monomers, Mₙ is the concentration of detergent micelles and n is the surfactant aggregation number, which was assumed to be 150 for MEGA-10 (M. T. Lee, A. Vishnyakov, A. V. Neimark, J. Phys. Chem. B 2013, 117, 10304; S. B. Sulthana, et al., Langmuir 2000, 16, 980). Based on this model, the association of detergent monomers into micelles is governed by Equation 2.

$${K}_{n} = \frac{{M}_{n}}{{M}^{n}} \qquad ( 2 )$$

In which Kₙ is the equilibrium constant which can be calculated from the detergent CMC (set at 5 mM for MEGA-10) (A. Walter, S. E. Suchy, P. K. Vinson, Biochim. Biophys. Acta—Biomembr. 1990, 1029, 67; M. T. Lee, A. Vishnyakov, A. V. Neimark, J. Phys. Chem. B 2013, 117, 10304; and S. B. Sulthana, et al., Langmuir 2000, 16, 980) through Equation 3.

$$C M C = {( n {K}_{n} )}^{- \frac{1}{n}} \qquad ( 3 )$$

TFF was modeled based on previously described mathematical expressions (I. S. Pires, A. F. Palmer, J. Memb. Sci. 2021, 618, 118712). Briefly, the system (sample container, tubes and internal filter contents) was assumed to be well mixed. Further, the total sample volume (V) was assumed to be constant such that the feed flow rate (F) and permeate flow rates (P) were equal. Both the detergent micelles (Mₙ) and saponin (S) were assumed to not permeate the membrane such that the only species in the permeate were detergent monomers whose concentration

$$( {C}_{M}^{perm} )$$

can be determined based on Equation 4.

$${C}_{M}^{p e r m} = M * ( 1 - {R}_{M} ) \qquad ( 4 )$$

In which R_{M} is the retention factor of surfactant monomers for each membrane which was set to be 10% for the 10 kDa membrane and 5% for the 100 kDa membrane.

To determine the concentration of material overtime in the TFF system, a mass balance can be done yielding Equation 5.

$$\frac{d {C}_{i}^{vessel}}{d t} = ( {C}_{i}^{feed} - {C}_{\dot{t}}^{p e r m} ) * F / V \qquad ( 5 )$$

In which

$${C}_{i}^{v e s s e l} , {C}_{i}^{feed} and {C}_{i}^{p e r m}$$

are concentration of species i in the system, feed and permeate, respectively, and t is the process time. Equation 5 may be simplified by normalizing the processing time by the characteristic time for a diafiltration volume (τ=V/P=V/F) yielding Equation 6.

$$\frac{d {C}_{i}^{v e s s e l}}{d {t}_{D}} = ( {C}_{i}^{f e e d} - {C}_{i}^{p e r m} ) \qquad ( 6 )$$

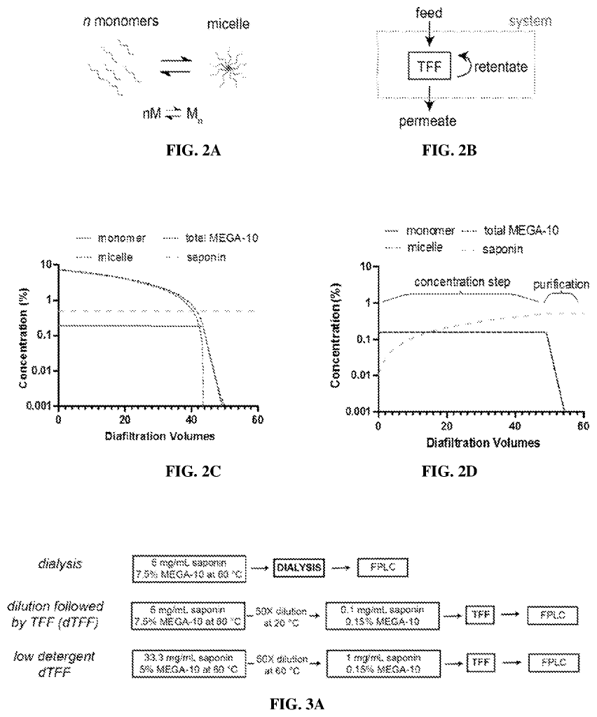

Combining Equations 2, 4 and 6 yields a differential-algebraic system of equations (DAE) (FIG. 2C) that may be solved using MATLABs ode15s function. The concentration step for the 100 kDa membrane model was modeled as a system in which the diluted material was fed into the system until reaching the initial saponin concentration. Purification was modeled by feeding in buffer (i.e., concentration of all species in feed was zero).

Quantification of particle composition and components. QS-21 SMNP particles were characterized via Reverse-Phase High Pressure Liquid Chromatography (RP-HPLC) to quantify each of its components. SMNPs were diluted 10× in 9:1 (v/v) 2-propanol:chloroform then separated on either a Jupiter C4 column (5 μm particles, 300 Å—Phenomenex P/N: 00G-4167-E0) or an Accucore C8 column (ThermoFisher). Detection was performed via low temperature evaporative light scattering detector (ELSD) on an ELSD-LT III (Shimadzu). Gradient times and operating conditions are shown in Table 1.

TABLE 1
RP-HPLC conditions and gradient

| Column | Jupiter C4 | | Accucore C8 | |
|---|---|---|---|---|
| Column Temperature | 40° C. | | 40° C. | |
| ELSD Temperature | 40° C. | | 40° C. | |
| Aqueous Phase (A) | 0.1% TFA Water | | 0.1M TEAA Water | |
| Organic Phase (B) | 0.1% TFA | | 0.1M TEAA 2- | |
| | Acetonitrile | | propanol | |
| Flow Rate | 1 ml/min | | | |
| Gradient | Time | % B | Time | % B |
| | 0 | 30 | 0 | 5 |
| | 5 | 30 | 5 | 5 |
| | 13 | 45 | 10 | 40 |
| | 17 | 45 | 15 | 40 |
| | 20 | 70 | 20 | 55 |
| | 27 | 85 | 30 | 55 |
| | 31 | 85 | 32.5 | 65 |
| | 41 | 95 | 36 | 65 |
| | 46 | 95 | 40 | 85 |
| | 47 | 30 | 45 | 85 |
| | 51 | 30 | 50 | 90 |
| | | | 70 | 95 |
| | | | 75 | 95 |
| | | | 75.05 | 5 |
| | | | 80 | 5 |

Animal Studies: Mouse experiments were performed at Massachusetts Institute of Technology (MIT). All experimental procedures were approved by Institutional Animal Care and Use Committees (IACUCs). Experiments were done using sex- and age-matched female mice between 6 and 12 weeks of age. C57Bl/6J mice (strain 000664) and BALB/c (strain 000651) were purchased form the Jackson Laboratory. For immunizations, mice were injected with 2 μg of recombinant N332-GT2 HIV Env gp140 trimer antigen and 5 μg of SMNP, subcutaneously (s.c.) bilaterally with half of the dose administered on each side of the tail base. Serum was collected 2-6 weeks after dosing and characterized for presence of antibodies against N332-GT2 trimer via enzyme-linked immunosorbent assays (ELISAs) as previously described (M. Silva, et al., Sci. Immunol. 2021, 6).

### Results

Dilution of Saponin and Lipids in Detergent Micelles Allows for Self-Assembly of ISCOMs without Compromising TFF Efficiency

Production of ISCOMs through dialysis is typically performed using low molecular weight cutoff (MWCO) membranes (e.g., 10 kDa). These membranes allow for detergent monomers (<1 kDa) to permeate but retain detergent micelles containing ISCOM components, which persist until the drop in detergent concentration allows the lipids and saponin self-assemble into their cage-like structure. Accordingly, as a first attempt for scalable manufacturing of ISCOMs, an attempt was made to process a solution of Quil-A-based ISCOM components (Quil-A saponin, cholesterol, and 1,2-dipalmitoyl-sn-glycero-3-phosphocholine [DPPC]) in MEGA-10 micelles through a 10 kDa MWCO hollow fiber TFF filter. However, as most of the detergent was in micellar form, filtration was ineffective with high operating pressures and low permeate fluxes due to the high concentration of material. This observation may explain why prolonged dialysis times are reported as necessary for ISCOM synthesis (3-5 days). As an alternative, it was tested to see whether ISCOM particles could be formed by diluting the micellar solution prior to TFF, such that high MWCO membranes could be used to facilitate detergent removal (FIG. 1A). In order to validate this idea, the equilibrium of detergent micelles and ISCOMs was first studied by characterizing a 2% MEGA-10 micelle sample with or without added ISCOM components via dynamic light scattering (DLS). As shown in FIG. 1B, at high concentration of detergent (2%), lipid/saponin/MEGA-10 mixtures showed particle sizes indicative of material being largely localized in MEGA-10 micelles. However, when diluted below the detergent CMC (~0.2-0.1% for MEGA-10), the detergent micelles disassembled into monomers such that DLS measurements either detected the detergent monomers (for the surfactant-only solution) or ISCOMs (40 nm particles) (A. Walter, S. E. Suchy, P. K. Vinson, Biochim. Biophys. Acta—Biomembr. 1990, 1029, 67). U.S. Pat. No. 5,679,354 discloses dilution but fails to mention the importance of control dilution step.

This result indicated that it is possible to generate ISCOMs prior to TFF by diluting the detergent mixture to below its CMC. This process was repeated using the same initial solution composition that has been used for dialysis-based ISCOM preparation (5 mg/mL ISCOM components in 7.5% MEGA-10), and DLS analysis of solutions rapidly diluted to different final concentrations was carried out, aiming to determine the optimal dilution conditions for ISCOM formation (FIG. 1C). Three regions of solution behavior were identified as a function of detergent final concentration: (1) At high MEGA-10 concentrations (>1%), micelles dominated with sizes of ~10 nm that did not change after overnight incubation. (2) At intermediate MEGA-10 concentrations (1%-0.2%), both micelles and ISCOMs were present, and DLS measured intermediate sizes between micelles and ISCOMs (10-40 nm), with minor increases in particle size after overnight incubation indicating the formation of ISCOMs. (3) At low MEGA-10 concentrations (<0.2%), the low micelle content due to proximity to the detergent CMC led to formation of larger species (~60 nm) that tended to condense into the desired ~40 nm diameter size of ISCOMs after overnight incubation.

Given that ISCOMs could be generated by diluting the detergent mixture to below its CMC, it was explored to see if it would be feasible to process the pre-formed ISCOMs via TFF to remove the detergent. It is generally preferable to operate TFF at high concentrations to increase total mass of impurities removed per unit volume in the permeate. Thus, to further explore whether dilution prior to TFF would affect filtration performance, a simple mathematical model was developed to determine how many diafiltration volumes (i.e., total permeate volume relative to sample volume) would be required to remove 7.5% of MEGA-10 through TFF from 5 mg/mL of saponin. Two scenarios were explored—constant volume diafiltration on a 10 kDa membrane with the high detergent concentration mixture (resembling the dialysis procedure) or dilution of the detergent mixture to below its CMC followed by a concentration step and constant volume diafiltration on a 100 kDa membrane. The detergent monomer to micelle equilibrium was represented through the closed-association model (FIG. 2A) and TFF was modeled based on previously described mathematical expressions derived from mass balances on the TFF system (FIG. 2B). The resulting set of differential algebraic equations (see Methods) were solved in MATLAB to determine the concentration of each species during TFF processing.

When processed through TFF on a 10 kDa membrane, the 7.5% MEGA-10 with 5 mg/mL saponin required approximately 50 diafiltration volumes to reduce the MEGA-10 content to 0.001% (FIG. 2C). While a 1000-fold reduction of a small MW impurity such as the detergent should be achieved in ~10 diafiltrations, the detergent-micelle equilibrium limited the monomer concentration in solution to the detergent CMC, limiting the concentration of MEGA-10 in the permeate. Therefore, even though a 50× dilution to 0.15% MEGA-10 was performed on the second case, the concentration of detergent monomers in the permeate was similar in both scenarios such that concentrating prior to TFF achieved the same level of MEGA-10 reduction in ~55 diafiltration volumes (FIG. 2D). In this scenario, 49 diafiltrations volumes were performed for concentrating the saponin and 6 for purification. Although this indicated a slight increase in total buffer processing by diluting the sample prior to processing, the 100 kDa membranes are shown to have around 10-fold higher permeate fluxes relative to 10 kDa membranes, indicating that the dilution prior to TFF processing could achieve purification in a significantly shorter amount of time (Repligen, Spectrum® mPES Hollow Fiber Filter Modules, Rancho Domingues, 2021). Further, as mentioned, the 7.5% MEGA-10 solution was impractical to process via TFF due to the high concentration of detergent. Based on these observations, a strategy to develop a complete ISCOM production process by first diluting the MEGA-10/lipids solution below the detergent CMC to generate ISCOMs, followed by use of a large MWCO membrane to easily concentrate the material and purify detergent monomers from ISCOMs was designed.

### Dilution Prior to Ultrafiltration Yields Biophysically Similar ISCOM and SMNP Particles Compared to Dialysis

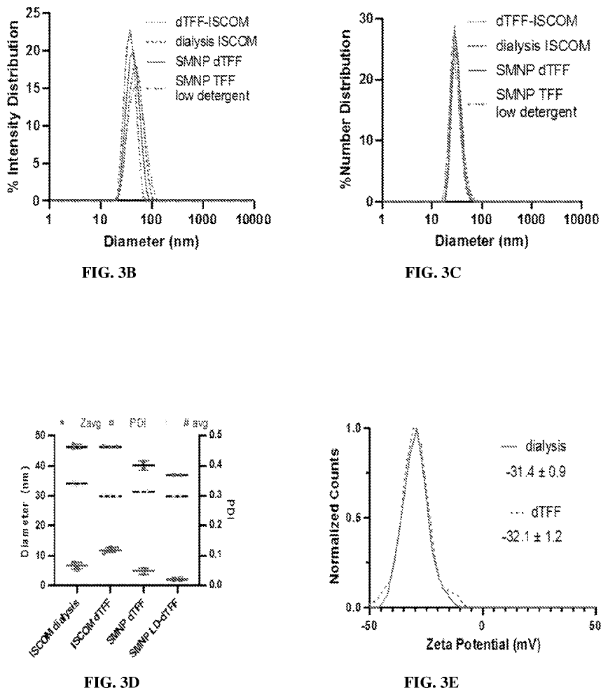

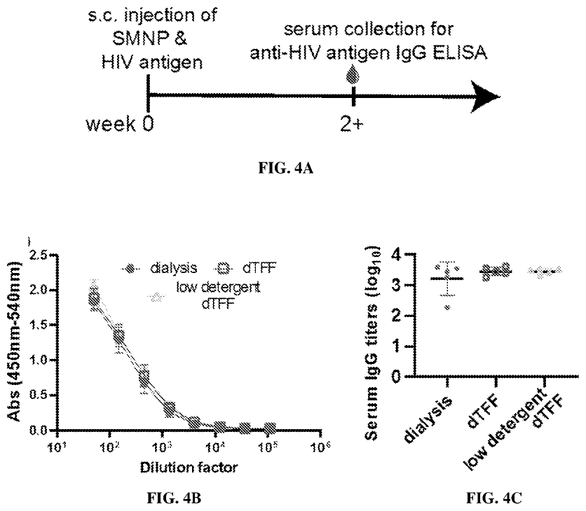

Next, ISCOMs and SMNP generated via dilution followed by TFF (dTFF) were studied to determine if they are structurally and biologically similar to particles formed by the standard dialysis protocol. All saponin and lipid components were solubilized in 20% MEGA-10 at 60° C. and mixed with PBS to generate a 5 mg/mL mg/mL solution of Quil-A saponin (together with the other ISCOM/SMNP components at the mass ratios indicated in FIG. 1A) in 7.5% MEGA-10 as previously described for dialysis-based ISCOM synthesis (Silva, et al., Immunol. 2021, 6.). This mixture was then either dialyzed or diluted to 0.15% MEGA-10 to induce ISCOM formation prior to TFF (FIG. 3A). In addition, to minimize the processing volume for TFF, a second TFF-based procedure was carried out where the total amount of detergent used for SMNP production was reduced 10-fold, by solubilizing 33.3 mg/mL of Quil-A saponin in 5% MEGA-10 at 60° C., then diluting the mixture to the same final concentration of MEGA-10 (0.15%) (FIG. 3A). After ISCOM formation, samples were processed on 100 kDa hollow fiber TFF membranes to concentrate particles to 1 mg/mL saponin then remove detergent monomers via continuous diafiltration for 10 diafiltration volumes.

After a FPLC purification employed for Quil-A formulations, ISCOMs or SMNP generated via either dialysis, dTFF, or low detergent-dTFF were indistinguishable based on DLS measurements (FIGS. 3B-3D). As SMNP requires incorporation of the negatively charged MPLA phospholipid, their zeta potential was also characterized and found no difference between the dialysis method or dTFF SMNPs (FIG. 3E). When assessed via TEM, all preparations yielded the characteristic cage-like morphology of ISCOMs or SMNP. Interestingly, it was noted that ISCOMs prepared by dialysis contained a small proportion of worm-like species that were not seen in the dTFF material, potentially indicating an improved robustness of ISCOM production via dTFF.

It was further evaluated whether any changes in the biological adjuvant activity of the SMNP preparations were observed in vivo by immunizing mice with a human immunodeficiency virus (HIV). Mice were injected with a mixture of the antigen and the different SMNP adjuvant formulations then their serum was collected to evaluate the anti-HIV antigen specific immunoglobulin G (IgG) antibody response (FIG. 4A).

The results from ELISA serum titer curves showed that immunization adjuvanted with Quil-A SMNP generated via dialysis, dTFF or low detergent dTFF had indistinguishable anti-HIV antigen IgG responses in mice (FIGS. 4B-4C).

### Generation of Scalable QS-21 SMNP Requires Control Over Dilution Rates to Generate ISCOMs

While Quil-A is widely used in many preclinical studies and is used in an approved equine vaccine, Quil-A is a mixture of more than 20 different saponin components (F. S. Oladunni, S. O. Oseni, L. Martinez-Sobrido, T. M. Chambers, Viruses 2021, 13, 1657; and D. Z. Wenbin Tuo, Nat. Prod. Chem. Res. 2015, 03). When separated via reverse-phase chromatography, the 21^{st} peak—termed QS-21—was found to be very potent without high toxicity making QS-21 a promising material for the development of saponin-based adjuvants for human use (C. R. Kensil, U. Patel, M. Lennick, D. Marciani, J. Immunol. 1991, 146). Indeed, QS-21 is used in the adjuvant formulation of two approved human vaccines—SHINGRIX® against herpes zoster by the US FDA and MOSQUIRIX® against malaria approved by the European Medicines Agency (P. Wang, Vaccines 2021, 9, 222). QS-21 is also used in a commercially available feline leukemia virus vaccine. It was previously shown that SMNP prepared by dialysis using QS-21 or Quil-A had similar adjuvant activity in vivo. In order to facilitate scalable manufacturing of QS-21 SMNP for human clinical trials, strategies were designed to use the standard dTFF protocol and the low detergent dTFF protocols developed above with Quil-A to generate QS-21 SMNP.

When replicating the process used for Quil-A SMNP synthesis with QS-21 (FIG. 5A), proper ISCOM formation was not observed. After solubilizing QS-21, cholesterol, MPLA, and lipid in MEGA-10 to generate a 7.5% MEGA-10 solution with 5 mg/mL QS-21, DLS measurements indicated the formation of species larger than ISCOMs (>60 nm), with the sample PDI (>0.2) indicating a polydisperse mixture upon dilution with PBS (FIGS. 5B-5C). After TFF processing, while the final products had the expected number average size of ~30 nm, the DLS intensity distribution showed a skew towards larger species (FIGS. 5D-5E). TEM revealed that the SMNP components did not properly interact as lipid particles and ring-like micelles were prevalent on the TEM micrographs either before or after TFF. Prior studies have shown that the ring-like micelles and worm-like micelles are primarily composed of saponin and cholesterol indicating that the lipidic components did not properly interact leading to self-assembly of two separate species (H.-X. Sun, Y. Xie, Y.-P. Ye, Vaccine 2009, 27, 4388, P. H. Demana, B. Berger, U. Vosgerau, T. Rades, N. M. Davies, J. Pharm. Pharmacol. 2010, 56, 573). Similarly, the low detergent dTFF product was found to contain ring-like micelles and lipid particles. Formation of rod-like structures in the low detergent dTFF product was also noted, which are also known to be favored when the mole fraction of cholesterol is increased in ISCOM formulations (H. L. Pham, P. N. Shaw, N. M. Davies, Int. J. Pharm. 2006, 310, 196). However, unlike the standard dTFF particle, almost no cage-like particles were seen on TEM of the low detergent dTFF QS-21 SMNP. These results indicate that QS-21 preparations prefer a higher amount of detergent to generate the desired cage-like SMNP particle structure, likely due to an incomplete solubilization of QS-21 into MEGA-10 micelles. While QS-21 is one of the more hydrophobic fractions contained in Quil-A, such significant formulation differences were not expected as QS-21 is known to form micelles of ~7 nm in aqueous solutions with solubility of up to 30 mg/mL in PBS (Quil-A also forms micelles and is soluble at >100 mg/mL) (C. R. Kensil, J. Y. Wu, S. Soltysik, Pharm. Biotechnol. 1995, 6, 525). This hydrophobicity originates from the fatty acid chain attached to the 4-position in the triterpenoid aglycone of saponins that is present in many of the saponin species of Quil-A (International Publication No. WO 2013/051994 by B. Morein).

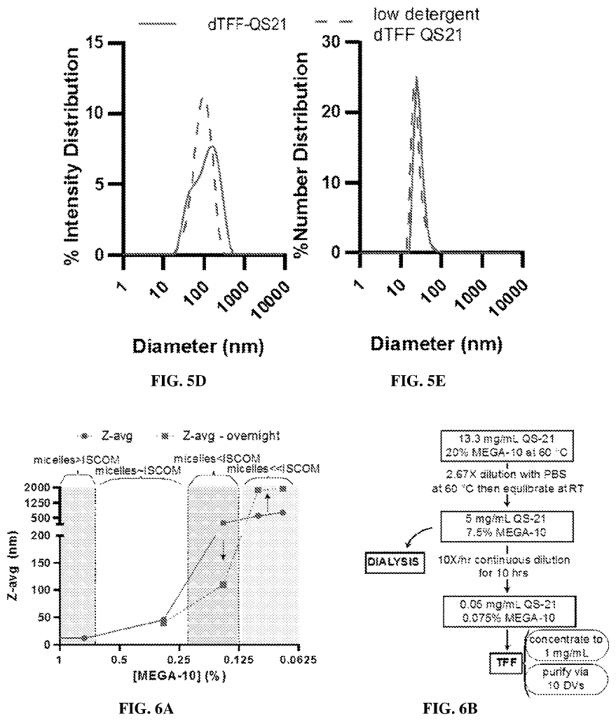

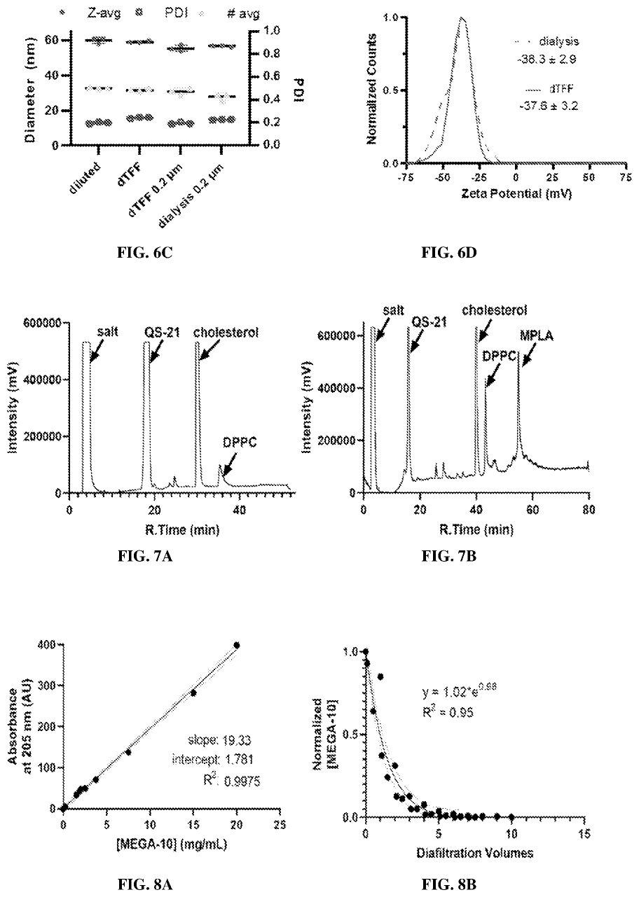

Based on these observations, the dilution behavior of the lipid/QS-21/MEGA-10 mixture was characterized, starting from the 5 mg/mL of QS-21 in 7.5% MEGA-10 and rapidly diluting to different endpoint MEGA-10 concentrations as was done for Quil-A. The “standard” dTFF protocol rather than the low-detergent method was the focus, as the standard procedure generated some cage-like particles whereas low detergent dTFF material consisted mostly of unwanted species. As shown in FIG. 6A, similar to the Quil-A ISCOMs, three regions of self-assembly were identified with lowering MEGA-10 concentration. However, unlike Quil-A samples which were capable of properly forming ISCOMs in the “low micelle concentration” region (<0.2% MEGA-10), QS-21 preparations rapidly diluted to these concentrations formed particles with initial mean sizes of more than 250 nm, which lowered to −100 nm after overnight incubation. Moreover, upon reducing MEGA-10 concentration further to less than −0.125% MEGA-10, we observed a fourth regime in which, instead of particles reorganizing during overnight incubation, aggregates were kinetically trapped due to the low micelle concentration, and further aggregated into micron-sized species upon overnight incubation. TEM micrographs revealed some cage-like particles in the preparations, but most of the sample was present in large lipid-saponin aggregates. Similar observations where presented in previous attempts to generate QS-21 ISCOMs (International Publication No. WO1992006710A1).

To overcome this limitation with QS-21 and better recapitulate the process of dialysis previously used to generate QS-21 SMNP, attempts were made to slowly dilute the QS-21 mixture containing 5 mg/mL QS-21, 7.5% MEGA-10 and the other SMNP components. This dilution was performed at a rate of 10-fold per hour, to gradually move from the starting solution into the “low micelle concentration” region (0.075%), with the goal of allowing the ISCOM components to reach their equilibrium assembly state before becoming trapped in their final structures as the MEGA-10 concentration dropped (FIG. 6B).

It has been observed that well-structured QS-21 SMNP particles could be formed and processed via dTFF using this slow continuous dilution approach (FIGS. 6C-6D). Size (FIG. 6C) and zeta-potential (FIG. 6D) measurements showed indistinguishable physical properties of the final products generated via either dTFF or dialysis. TEM imaging revealed a homogeneous cage-like particle morphology for dTFF QS-21 SMNP. The composition of particles prepared by the dTFF or dialysis process was also assessed using reverse phase HPLC (RP-HPLC). As shown in Table 2, the final composition of QS-21 SMNP particles generated dTFF closely mirrored the input 10:2:1:1 saponin:cholesterol:DPPC:MPLA (by mass) feed ratio added to the synthesis. Representative RP-HPLC chromatograms for QS-21 SMNPs with identified peaks are shown in FIGS. 7A-7B. Further, both methods led to high yields (70-80% by QS-21 content) as determined via RP-HPLC. Using either dialysis or dTFF preparation, no MEGA-10 was detectable via RP-HPLC, which agreed with permeate measurements indicating that most of the MEGA-10 was removed during the 10 diafiltration volumes which was confirmed through quantification of MEGA-10 in the permeate during TFF processing (FIGS. 8A-8B).

TABLE 2
Mass ratio of QS-21 SMNPs components relative
to saponin. Values indicate average of three
independent batches and standard deviation.

| | Component | | | |
|---|---|---|---|---|
| Stage | QS-21 | Cholesterol | DPPC | MPLA |
| starting mixture | 10 | 2 | 1 | 1 |
| dTFF | 10 | 1.95 ± 0.11 | 1.03 ± 0.15 | 1.02 ± 0.24 |
| yield (based on | 77 ± 3% | | | |
| QS-21) | | | | |

### QS-21 SMNP Generated Through Dilution and Ultrafiltration is Bioactive and Stable to Freezing

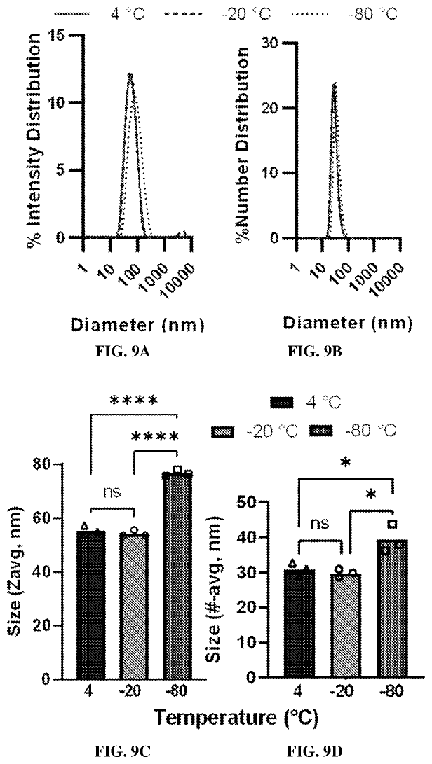

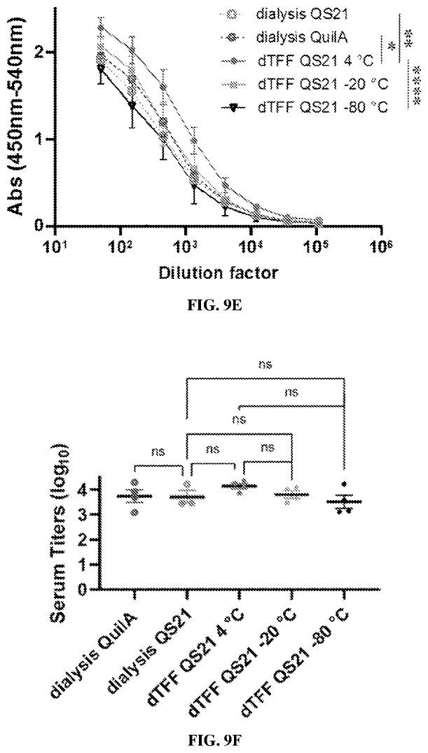

With a scalable process to generate clinical grade SMNP developed, experiments were designed to evaluate the bioactivity of the material and its stability when frozen at −20° C. or −80° C. to facilitate long term storage of material (FIGS. 9A-9F). In order to minimize sample formulation complexity, the stability of QS-21 SMNP frozen without additional cryoprotectants was first evaluated.

As shown in FIGS. 9A-9D, when frozen at −20° C., QS-21 SMNP particles did not exhibit any changes in their size even without the presence of cryoprotectants. Only when frozen at −80° C. was there a noticeable increase in particle size. Interestingly, it was found that QS-21 SMNP particles generated via dTFF were found to have improved adjuvant activity in vivo compared to either Quil-A or QS-21 SMNPs generated via dialysis based on their serum IgG titer curves (FIG. 9E). However, no differences were observed in the calculated serum IgG titers (FIG. 9F). Although QS-21 SMNP frozen at −80° C. showed some aggregation and a lower serum IgG titer curve than QS-21 SMNP at 4° C. (FIG. 9E), there was no difference in serum IgG titers for dTFF samples frozen at −20° C. or −80° C. compared to dialysis or dTFF samples stored at 4° C. (FIGS. 9E-9F). When assessed via TEM, particles kept at 4° C. or frozen were found to maintain the cage-like morphology and sample homogeneity. These results indicated that biologically active QS-21 SMNP could be generated through dTFF and that they may be readily frozen and transported at −20° C. without loss of its biological adjuvant properties.

### Staggered Discontinuous Dilution Improves Particle Quality and Facilitates Room Temperature QS-21 SMNP Synthesis

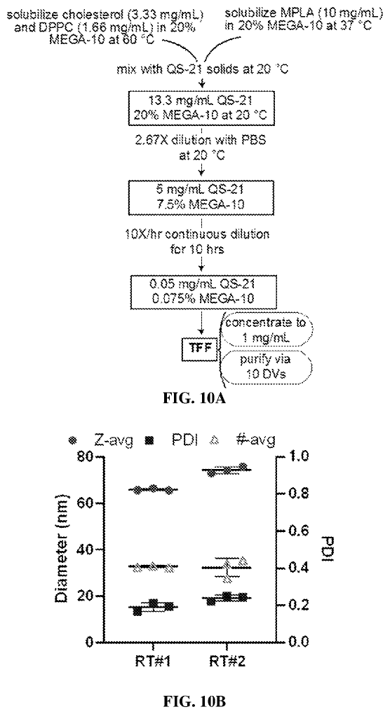

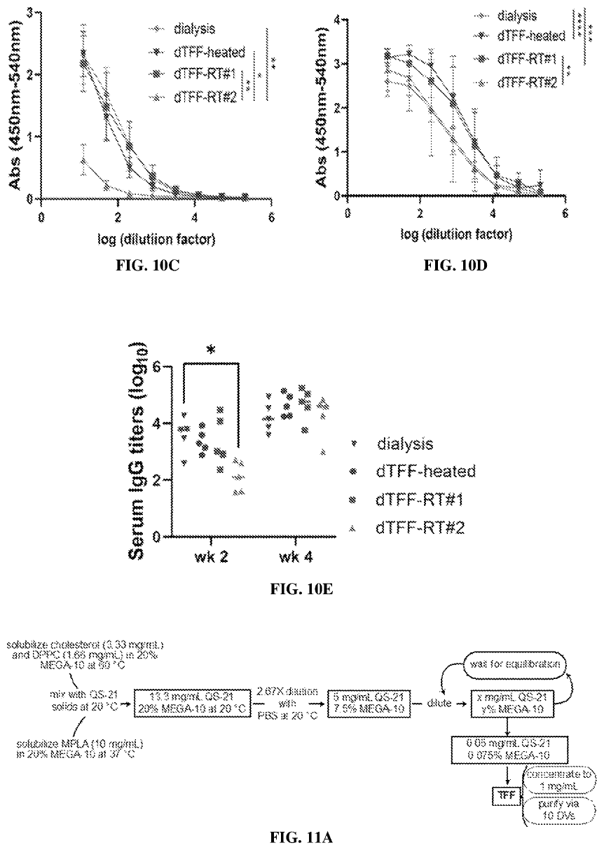

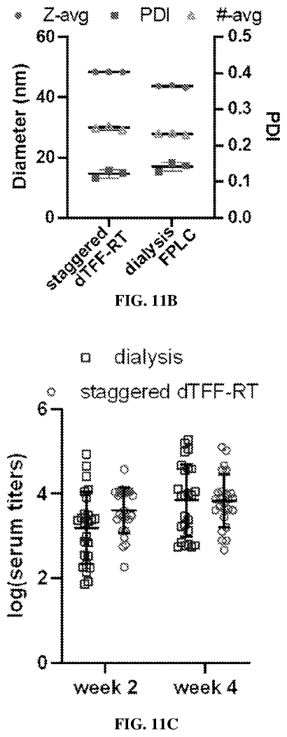

In the initial QS-21 SMNP process development, high temperature mixing at 60° C. was used to facilitates a fully dissolved initial lipid/MEGA-10 solution to be prepared. As QS-21 may undergo temperature-driven hydrolysis of its ester bond between the fucose and the fatty acid domain leading to inactivation (Kensil, et al. Pharm. Biotechnol. 1995, 6, 525), it was sought to remove the need for this high temperature mixing step. Heating was maintained for DPPC and cholesterol at 60° C. and MPLA at 37° C. to facilitate solubilization into MEGA-10 micelles as these materials should not be prone to rapid degradation in aqueous solutions (FIG. 10A). While it was possible to generate QS-21 SMNP by simply mixing all MEGA-10-solubilized components at room temperature followed by continuous dilution and TFF, the sample homogeneity was reduced relative to prior preparations, based on a Z-avg size larger than 60 nm and PDI greater than 0.2 (FIG. 10B).

In order to explore whether these inhomogeneities had an effect on the biological adjuvant activity of SMNPs, two in vivo room temperature dTFF SMNP batches were tested—one with highest homogeneity (dTFF-RT #1) and one with lowest (dTFF-RT #2). While dTFF-RT #1 batch was similar to heated dTFF SMNP or SMNP generated via dialysis, the preparation with largest Z-avg and PDI was found to have diminished serum IgG titers at week 2 compared to SMNP prepared by dialysis or the dTFF preparations (FIGS. 10C-1E). However, antibody responses reached similar levels by 4 weeks (FIGS. 10D-10E) and showed improved serum IgG curves from heated dTFF samples or the high-homogeneity room temperature dTFF relative to dialysis preparations. TEM micrographs analysis revealed a notable presence of lipid vesicles in these SMNP preparations, indicating incomplete complexing of saponin with phospholipids during the gradual dilution procedure.

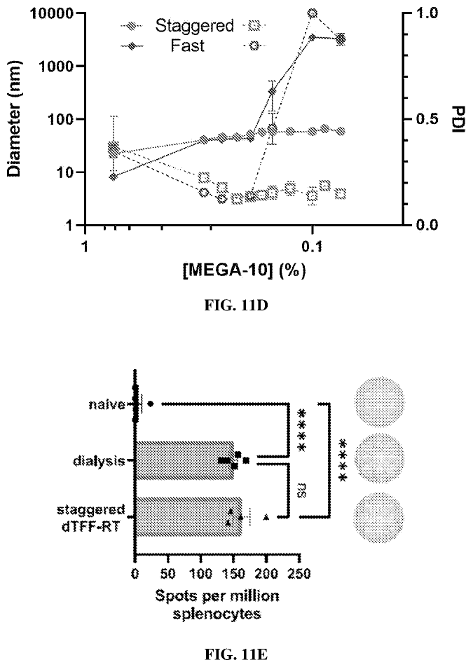

In order to overcome this issue, it was discovered that the sample homogeneity could be improved by diluting the MEGA-10/QS-21/lipid/cholesterol mixture in staggered steps instead of continuously (FIG. 11A, Table 3). It is believed that this improved self-assembly may be due to particles existing at the “intermediate” MEGA-10 concentrations in which micelles co-exist with ISCOMs. In the continuous dilution, the sample never reached equilibrium during its dilution as buffer was constantly added. In the staggered dilution, the sample was allowed to equilibrate after every dilution step such that a more complete complexing of saponin with phospholipids could be achieved and any potentially kinetically trapped species could be disassembled by the remaining micelles and formed into ISCOM structures. Notably, this new dilution protocol facilitated homogenous QS-21 SMNP preparations with Z-avg size and PDI similar to QS-21 SMNPs processed through SEC-FPLC after dialysis synthesis (FIG. 11B). Further, TEM micrographs revealed the desired prominent cage-like particle morphology. The same protocol facilitated synthesis of QS-21 ISCOMs (FIG. 11D) indicating that the MPLA component of SMNPs was not the driver for the aggregates formed via rapid dilution. FIG. 11D shows that staggered dilution facilitates proper assembly of QS-21 ISCOMs devoid of MPLA. A sample with 7.5% MEGA-10 and 5 mg/mL of QS-21-ISCOM components (QS-21, cholesterol and DPPC at a 5:1:1 mass ratio) was either rapidly diluted to final MEGA-10 concentrations or diluted via the staggered protocol of Table 3.

TABLE 3
Staggered dilution steps for QS-21 SMNP assembly.

| Dilution | Dilution Factor Relative to | Equilibration Time |
|---|---|---|
| Step | Starting Material | (min) |
| 1 | 10X | 10 |
| 2 | 25X | 45 |
| 3 | 30X | 20 |
| 4 | 35X | 20 |
| 5 | 40X | 10 |
| 6 | 45X | 10 |
| 7 | 50X | 10 |
| 8 | 60X | 10 |
| 9 | 75X | 10 |
| 10 | 85X | 10 |
| 11 | 100X | 10 |

To more completely assess any differences between dialysis-generated QS-21 SMNP and staggered-dTFF QS-21 SMNP, three independent batches of each were tested in vivo for their effect on seroconversion following immunization. No significant differences were observed on the particle adjuvant performance in mouse immunizations (FIG. 11C). To further validate that the SMNP from our TFF-based method was equivalent to the dialysis-based sample used in the preclinical studies, we performed an IFN-γ ELISpot assay and found that SMNP adjuvanted immunization led to a robust cellular response two weeks after vaccination which was of the same for both dialysis- and TFF-produced SMNP (FIG. 11E). These results confirm that the QS-21 SMNP could be synthesized at room temperature and processed via dTFF to yield biologically active and homogeneous QS-21 SMNP product. Notably, while most experiments presented here were performed at 5-15 mg scales of QS-21, this method has been successfully scaled to 50 mg and 500 mg batches of QS-21 SMNPs without processing changes except increasing TFF filter surface area and total processing volumes. Based on the experience gained from these experiments, a set of target Preferred Quality Attributes for SMNP were defined.

TABLE 4
Preferred Quality Attributes of SMNPs.

| Appearance | Colorless to whitish, clear to | |
|---|---|---|
| | slightly opalescent suspension | |
| QS-21:cholesterol:DPPC:MPLA | 10:2 ± 0.3:1 ± 0.3:1 ± 0.3 | |
| mass ratio | | |
| Particle size (Z_{avg}) | 40-80 | nm |
| Polydispersity index (PDI) | ≤0.25 | |
| Residual MEGA-10 | ≤1 | μg/mL |

| | |
|---|---|
| Particle morphology | Cage-like as characterized via |
| | negative stain TEM |

### SUMMARY

The next-generation ISCOM-like adjuvant termed SMNP has demonstrated promising preclinical adjuvant activity in mice, rabbits, and non-human primates. However, dialysis-based processing that is not easily adapted to GMP scale-up manufacturing was used to synthesize SMNP particles in these preclinical studies. While composition and buffer can affect particle morphology (Pham, et al., Int. J. Pharm. 310 (1-2) (2006) 196-202, doi.org/10.1016/j.ijpharm.2005.11.011; Walter, et al., Biochimica et Biophysica Acta (BBA)—Biomembranes 1029 (1) (1990) 67-74, doi.org/10.1016/0005-2736(90)90437-S; Kensil, et al., Pharm. Biotechnol. 6 (1995) 525-541, doi.org/10.1007/978-1-4615-1823-5_22.), the disclosed results demonstrate that the dilution rate is an important process parameter for proper assembly of QS-21 based ISCOMs. In addition to improved sample homogeneity, the controlled self-assembly of ISCOMs or SMNP through the method presented here may facilitate use of saponin fractions previously found to be very potent but that were difficult to generate into the cage-like particles. For example, when initially attempted to generate ISCOMs with the QS-21 fraction, they were found to form into >1 μm particles with some cage-like structures present similar to rapidly diluted samples (FIG. 6A) (K. G. F. Anne, S. Arjen, V. D. W. Gerrit, B. E. Coen, Immunogenic Complexes, In Particular ISCOMS, WIPO, Netherlands, 1991). Further, other Quil-A fractions such as QHB and QHC which are in formulations for clinical development were reported to have a lower ISCOM forming ability (U.S. Pat. No. 6,352,697 by C. J. COOPER). This lower propensity to form ISCOMs may originate from the higher hydrophobicity of these fractions, which is shown here may be overcome by increasing residence time at “intermediate” detergent concentrations required for proper ISCOM self-assembly (M. Bror, L. Karin, Pharmaceutical Carrier, WIPO, Sweden, 1992). Importantly, SMNP elicited higher antibody titers as well as higher antigen-specific CD4+ T cell production of IFN-γ and IL-21 compared to the potent liposomal saponin/MPLA adjuvant ASO1B (Silva, et al., Sci. Immunol. 6 (66) (2021), doi.org/10.1126/sciimmunol.abf1152.). These results indicate that the unique cage-like assembly of ISCOMs may play an important role in maximizing adjuvant activity, such that processes for its production need to ensure preservation of the particle structure. The methods presented here for controlled lipid self-assembly may also have utility for the synthesis of other soft matter assemblies of interest for broad applications in medicine and beyond.

In this work, detailed protocols are provided to produce ISCOMs or SMNP via a tangential flow filtration (TFF) process suitable for scalable synthesis and Good Manufacturing Practice (GMP) production of clinical-grade adjuvants. SMNP or ISCOM components were solubilized in micelles of the surfactant MEGA-10, then diluted below the critical micelle concentration (CMC) of the surfactant to drive controlled ISCOM self-assembly. Assembly of ISCOM/SMNP particles using the purified saponin QS-21 used in clinical-grade saponin adjuvant was found to require controlled stepwise dilution of the initial micellar solution, to prevent formation of undesirable kinetically-trapped aggregate species. A preferred protocol gave yields of ~77% of QS-21 and the final SMNP particle composition mirrored the feed ratios of the components. Further, samples were highly homogeneous with comparable quality to that of material prepared at lab scale by dialysis and purified via size-exclusion chromatography. This protocol may be useful for clinical preparation of ISCOM-based vaccine adjuvants and therapeutics.

Pires, et al., “Controlled lipid self-assembly for scalable manufacturing of next-generation immune stimulating complexes,” Chemical Engineering Journal, Volume 464, 15 May 2023, 142664, 11 pages and all of the associated supplemental materials, doi.org/10.1016/j.cej.2023.142664, is specifically incorporated by reference herein in its entirety.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize or be able to ascertain using no more than routine experimentation, many equivalents to the specific embodiments of the invention described herein. Such equivalents are intended to be encompassed by the following claims.

1. A method of making nanocage particles comprising the steps of
(a) solubilizing and/or mixing one or more components of lipids, cholesterol, saponin and optionally an additional adjuvant, in the presence of a detergent to form detergent micelles,
(b) controllably dissociating the detergent micelles into monomers; and
(c) removing the detergent from the mixture,
wherein the step of (b) and/or (c) promotes self-assembly of the one or more components of lipids, cholesterol, saponin and optionally an additional adjuvant into a dispersion of nanocage particles.

2. The method of claim 1, wherein step (b) comprises reducing the concentration of detergent to achieve a desired concentration of the detergent.

3. A method of making nanocage particles comprising the steps of
(a) solubilizing and/or mixing one or more components of lipids, cholesterol, saponin and optionally an additional adjuvant, in the presence of a detergent to form detergent micelles,
(b) reducing the concentration of detergent to achieve a desired concentration of the detergent; and
(c) removing the detergent from the mixture,
wherein the step of (b) and/or (c) promotes self-assembly of the one or more components of lipids, cholesterol, saponin and optionally an additional adjuvant into a dispersion of nanocage particles.

4. The method of claim 3, wherein step (b) comprises a step of dilution of the mixture of (a) to achieve a desired concentration of the detergent before the step (c).

5. The method of claim 4, wherein the dilution step is a rapid dilution step achieving a desired concentration of the detergent in less than 30 minutes, less than 20 minutes, less than 10 minutes, or less than 5 minutes, or less than one minute.

6. The method of claim 4, wherein the dilution step is a continuous dilution step achieving a desired concentration of the detergent adding buffer dropwise to the solution over a period of between about 1 hour and about 24 hours.

7. The method of claim 6, wherein the continuous dilution step is carried out at a rate to achieve 10-fold dilution per hour for 10 hours.

8. The method of claim 6, wherein the dilution step is a staggered discontinuous dilution step achieving a desired concentration of the detergent via stepwise addition of buffer, wherein after each step of adding buffer, the mixture is allowed to equilibrate prior to the next step of adding buffer,
optionally the staggered discontinuous dilution step is performed diluting in 10-fold steps relative to the initial volume and allowing samples sufficient time to equilibrate mixture, before performing the next dilution step.

9. The method of claim 4, wherein step (b) comprises increasing the ionic strength of the mixture, changing the temperature, adding one or more detergent-depleting agents to change the detergent micelle-monomer equilibrium.

10. The method of claim 4, wherein the desired concentration of the detergent is about or below the critical micelle concentration (CMC) of the detergent.

11.-13. (canceled)

14. The method of claim 4, wherein the step of removing the detergent from the mixture is carried out via dialysis, centrifugation, filtration, or combinations thereof.

15.-20. (canceled)

21. The method of claim 4, wherein (i) the mass ratio of lipid:additional adjuvant:sterol:saponin is 1:1:2:10, (ii) the mass ratio of lipid:sterol:saponin is 1:1:5, (iii) or a variation thereof wherein the mass ratio of lipid, additional adjuvant, sterol, saponin or any combination thereof is increased or decreased by any value between about 0 and about 3.

22.-40. (canceled)

41. The method of claim 4, wherein the lipid is DPPC, the additional adjuvant is a natural or synthetic MPLA, the sterol is cholesterol, and the saponin is Quil A.

42. (canceled)

43. The method of claim 4, wherein the lipid is DPPC, the additional adjuvant is a natural or synthetic MPLA, the sterol is cholesterol, and the saponin is QS-21.

44. The method of claim 43, wherein the mass ration of QS-21:cholesterol:DPPC:MPLA is 10:2±0.3:1±0.3:1±0.3.

45. The method of claim 43, wherein the DPPC:MPLA:cholesterol:Q-21 are in a mass ratio of 1:1:2:10.

46. The method of claim 44, wherein the mixture is a colorless to whitish, clear to slightly opalescent suspension; the particle size (Z_{avg}) is 40 nm-80 nm; the polydispersity index (PDI) is ≤0.25; residual detergent, optionally MEGA-10, is ≤1 μg/mL; and the particle morphology is cage-like or any combination thereof.

47. (canceled)

48. A nanocage particle made according to the method of claim 4.

49. A pharmaceutical composition comprising a plurality of the nanocage particles of claim 48 and a pharmaceutical carrier.

50.-62. (canceled)

63. A kit comprising a plurality of the nanocage particles made according to the method of claim 4 in a lyophilized or dried form, or suspended in a pharmaceutically acceptable carrier.

64.-65. (canceled)
